(19) United States
(12) Patent Application Publication
(10) Pub. No.: US 2014/0186308 A1
(43) Pub. Date: Jul. 3, 2014

(54) COMPOSITIONS FOR DIRECTING ADIPOSE-DERIVED STEM CELLS TO A CHONDROGENIC DIFFERENTIATION AND METHODS OF USE THEREFOR

(71) Applicant: The Salk Institute for Biological Studies, La Jolla, CA (US)

(72) Inventors: Juan Carlos Belmonte, La Jolla, CA (US); Senyon Choe, Solana Beach, CA (US); Sheng-Lian Yang, San Diego, CA (US); Macarena Peran, Albolote (ES)

(73) Assignee: The Salk Institute for Biological Studies, La Jolla, CA (US)

(21) Appl. No.: 14/101,827

(22) Filed: Dec. 10, 2013

Related U.S. Application Data
(60) Provisional application No. 61/735,271, filed on Dec. 10, 2012.

(51) Int. Cl. A61K 35/12

(52) U.S. Cl. CPC A61K 35/35
USPC 424/93.7; 435/375

(57) ABSTRACT

Composition and methods are provided to stimulate the proliferation and differentiation of pluripotent stem cells into chondrocytes. The method comprises the steps of contacting the pluripotent stem cells with an AB2 chimeric polypeptide under conditions suitable for grown and proliferation of the cells.

### CROSS REFERENCE TO RELATED APPLICATIONS

This application is a U.S. nonprovisional application, which claims priority to and the benefit of U.S. provisional patent application Ser. No. 61/735,271, filed Dec. 10, 2012. The entire contents of the aforementioned patent application is incorporated herein by this reference.

### BACKGROUND

Degenerative diseases associated with age represent a major health challenge in developed countries. In particular, the intrinsic properties of cartilage tissue, its avascular nature, and deficient self-repair capacity imply that cartilage degeneration or associated injures may become a chronic problem, to which no effective solution has yet been found. In fact, over 40 million people in the United States suffer from conditions related to cartilage damage. Moreover, cartilage degeneration causes not only joint pain and dysfunction, but also increases the percentage of people with limited mobility.

Cartilage is avascular connective tissue formed by only one cell type, the chondrocyte, trapped in the extracellular matrix composed mainly of collagen fibers, aggrecan (a large aggregating proteoglycan) and water. In fact, many efforts are currently being made to create cartilage in laboratories by combining novel biomaterials, growth factors and cells.

Clinical treatment for articular cartilage injury includes autologous cell injections of primary chondrocytes expanded in vitro. However, the dedifferentiation of the autologous chondrocytes in culture and the small number of cells that can be obtained limit their clinical applications only to small injuries. Alternative cell sources for cartilage tissue engineering are mainly embryonic stem cells and adult stem cells. Mesenchymal stem cells derived from bone-marrow (MSCs) and adipose tissue (ASCs) have shown significant chondrogenic potential. Importantly, ASCs can be easily harvested, expanded in vitro and are relatively abundant in comparison with MSC. Therefore, ASCs could be an ideal cell type for generating a large number of functional chondrocytes.

The transforming growth factor-β (TGF-β) superfamily is comprised of almost forty ligands responsible for numerous cellular processes including early embryonic development, tissue patterning and homeostasis, bone formation, wound healing and fibrosis. These proteins signal through the simultaneous interaction with one of the 7 type I and one of the 5 type II TGF-β receptor (TGFR-β) kinases. The signalling requires the hetero-dimerization and subsequent activation of both types I and II receptors through the binding of their TGF-β ligands. Interestingly, different TGF-β ligands, such as Activin as well as several isoforms of bone morphogenetic protein (BMPs), TGF-β1 or TGF-β3, have been shown to promote chondrogenesis. Activin A exhibits high affinity for type II receptors and signals through SMAD2/3 transcription factors, while BMP2 possesses higher affinity for type I receptors and signals through SMAD1/5/8 transcription factors.

A need exists for methods of generating large numbers of chondrogenic cells for autologous transplant.

### SUMMARY

In accordance with one embodiment compositions and methods are provided for differentiating a large number of human adipose stromal cells towards chondrocytes in a reproducible and efficient manner. The method comprises the steps of contacting adipose stromal cells with one or more Activin/BMP-2 Chimeric Ligands (AB2 ligands) to scalably enhance the formation of chondrocytes from the adipose stromal cells. Chondrocytes obtained by the herein disclosed induction methods are anticipated to have novel and important implications for cell replacement therapies in cartilage repair clinical protocols.

In one aspect, the invention provides a composition containing a purified chimeric polypeptide that is any one or more of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab; and a purified population of adipose stromal cells.

In another aspect, the invention provides a method of increasing the expression of chondrogenic proteins in purified adipose stromal cells, the method involving contacting a purified population of adipose stromal cells with a purified chimeric polypeptide that is any one or more of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab under conditions conducive for growth of the cells, thereby increasing expression of chondrogenic proteins.

In yet another aspect, the invention provides an adipose stromal cell having increased expression of type II collagen, Sox9, and Aggrecan relative to a control, where the adipose stromal cell is capable of differentiating into an articular chondrogenic lineage. In one embodiment, the cell has reduced expression of type I collagen relative to a control.

In yet another aspect, the invention provides a method for treating a subject having cartilage damage or degeneration the method involving administering to the subject an adipose stromal cell having increased expression of type II collagen, Sox9, and Aggrecan relative to a control, where the adipose stromal cell is capable of differentiating into an articular chondrogenic lineage. In one embodiment, the cell is injected at a site where chondrocyte formation is desired.

In yet another aspect, the invention provides a kit for inducing the formation of chondrocytes, the kit containing a purified chimeric polypeptide that is any one or more of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab; a suitable cell culture media; and instructional material.

In still another aspect, the invention provides a composition containing a purified chimeric polypeptide containing the sequence of MQAKHKQRKRLKSSCKRHPLYVDFSDVGWNDWIIAPSGYHANYCEGECPSHIAGTS GSSLSFHSTLVNHYRMRGHSPFANLKSCCVPTKLRPMSMLYYDDGQNVVLKNYQD MIVEECGCS; and culture media containing Dulbecco's modified Eagle's medium with 10% fetal bovine serum.

In various embodiments of the above aspects or any other aspect of the invention delineated herein, the purified chimeric polypeptide contains the sequence MQAKHKQRKRLKSSCKRHPLYVDFSDVGWNDWIIAPSGYHANYCEGECPSHIAGTS GSSLSFHSTLVNHYRMRGHSPFANLKSCCVPTKLRPMSMLYYDDGQNVVLKNYQD MIVEECGCS. In one embodiment, the chimeric polypeptide is combined with or linked to a biocompatible polymer (e.g., collagen, fibronectin, polyglycol acid (PGA), polylactic acid (PLA) and a co-polymer of PGA and PLA). In various embodiments, a cell of the invention is grown or provided in culture media containing Dulbecco's modified Eagle's medium with 10% fetal bovine serum. the chimeric polypeptide is selected from the group consisting of 1b2a3b4b5a6a (AB204), 1b2a3b4b5b6a (AB215) and 1b2a3a4a5a6ab (AB235). In various embodiments of the above aspects or any other aspect of the invention delineated herein, the purified chimeric polypeptide comprises the sequence MQAKHKQRKRLKSSCKRHPLYVDFSDVGWNDWIIAPSGYHANYCEGECPSHIAGTS GSSLSFHSTLVNHYRMRGHSPFANLKSCCVPTKLRPMSMLYYDDGQNVVLKNYQD MIVEECGCS. In various embodiments of the above aspects or any other aspect of the invention delineated herein, the composition further comprises an extracellular matrix protein or glycoprotein. In various embodiments of the above aspects or any other aspect of the invention delineated herein, the chondrogenic proteins are selected from the group consisting of collagen II, aggrecan and Sox9. In various embodiments of the above aspects or any other aspect of the invention delineated herein, the method is carried out in vivo or in vitro. In various embodiments of the above aspects or any other aspect of the invention delineated herein, method further comprises administering the compostion to a subject. In various embodiments of the above aspects or any other aspect of the invention delineated herein, purified adipose stromal cells are derived from the subject. In other embodiments, the composition is injected into the subject at a site where chondrocyte formation is desired. In various embodiments of the above aspects or any other aspect of the invention delineated herein, adipose stromal cells are contacted with AB235 having the sequence M Q A K H K Q R K R L K S S C K R H P L Y V D F S D V G W N D W I I A P S G Y H A N Y C E G E C P S H I A G T S G S S L S F H S T L V N H Y R M R G H S P F A N L K S C C V P T K L R P M S M L Y Y D D G Q N V V L K N Y Q D M I V E E C G C S. In other embodiments, the contact increases type II collagen expression by about 79 fold, Sox9 expression by about 14 fold, and Aggrecan expression by about 20-fold relative to control.

In accordance with one embodiment a composition comprising a mixture of purified adipose stromal cells and one or more Activin/BMP-2 chimeric ligands is provided for stimulating the production of functional chondrocytes from adipose stromal cells. In accordance with one embodiment the compositions comprise adipose stromal cells and AB2 ligands, optionally combined with a biocompatible polymer. The purified adipose stromal cells are typically primary cells that are purified from mammalian tissues, including for example, from adipose tissue. In one embodiment the cells are held within a collagen/fibronectin matrix.

The present disclosure further describes a method of upregulating the expression of chondrogenic-related genes in purified adipose stromal cells. The method comprises the steps of contacting a purified population of adipose stromal cells with one or more Activin/BMP-2 chimeric ligands under conditions conducive to cell proliferation (e.g., suitable culture media, temperature and pH), resulting in the differentiation of adipose stromal cells to chondrocytes.

The present disclosure further encompasses a kit for inducing the formation of chondrocytes from pluripotent stem cells. In one embodiment the kit comprises an AB2 purified chimeric polypeptide selected from the group consisting of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab; a suitable cell culture media; and instructional material. The kit may comprise additional components and labware for use in expanding the initial populations of stromal cells, as well as components for administering the cells to a patient. In one embodiment the kit further comprises components for forming a biocompatible matrix to be used in conjunction with the cells.

### Definitions

In describing and claiming the invention, the following terminology will be used in accordance with the definitions set forth below.

The term “about” as used herein means greater or lesser than the value or range of values stated by 10 percent, but is not intended to limit any value or range of values to only this broader definition. Each value or range of values preceded by the term “about” is also intended to encompass the embodiment of the stated absolute value or range of values.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes prophylaxis of the specific disorder or condition, or alleviation of the symptoms associated with a specific disorder or condition and/or preventing or eliminating said symptoms.

As used herein the term “biologically derived agents” include one or more of the following: bone (autograft, allograft, and xenograft) and derivates of bone; cartilage (autograft, allograft, and xenograft), including, for example, meniscal tissue, and derivatives; ligament (autograft, allograft, and xenograft) and derivatives; derivatives of intestinal tissue (autograft, allograft, and xenograft), including for example submucosa; derivatives of stomach tissue (autograft, allograft, and xenograft), including for example submucosa; derivatives of bladder tissue (autograft, allograft, and xenograft), including for example submucosa; derivatives of alimentary tissue (autograft, allograft, and xenograft), including for example submucosa; derivatives of respiratory tissue (autograft, allograft, and xenograft), including for example submucosa; derivatives of genital tissue (autograft, allograft, and xenograft), including for example submucosa; derivatives of liver tissue (autograft, allograft, and xenograft), including for example liver basement membrane; derivatives of skin tissue; platelet rich plasma (PRP), platelet poor plasma (PPP), bone marrow aspirate, demineralized bone matrix, whole blood, fibrin, and blood clot. Purified ECM and other collagen sources are also intended to be included within “biologically derived agents.”

As used herein the term “biocompatible polymers” is intended to include both synthetic polymers and biopolymers (e.g. collagen), and a “biocompatible polymer matrix” refers to a mass formed by such polymers. Examples of biocompatible synthetic polymers include: polyesters of [alpha]-hydroxycarboxylic acids, such as poly(L-lactide) (PLLA) and polyglycolide (PGA); poly-p-dioxanone (PDO); polycaprolactone (PCL); polyvinyl alcohol (PVA); polyethylene oxide (PEO); polymers disclosed in U.S. Pat. Nos. 6,333,029 and 6,355,699; co-polymer or a mixture of polymers and/or co-polymers such as polymer networks of poly(acrylamide-co-ethylene glycol/acrylic acid) or poly(lysine-(lactide-ethylene glycol)) or any other biocompatible polymer, co-polymer or mixture of polymers or co-polymers that are utilized in the construction of prosthetic implants or prosthetic implant coatings. Examples of commercially available synthetic biocompatible polymers include Prolene™, Vicryl™, Mersilene™, and Panacryl™. In addition, as new biocompatible, bioresorbable materials are developed, it is expected that at least some of them will be useful materials from which orthopaedic devices may be made. It should be understood that the above materials are identified by way of example only, and the present invention is not limited to any particular material unless expressly called for in the claims.

As used herein the term “biocompatible inorganic materials” include materials such as hydroxyapatite, all calcium phosphates, alpha-tricalcium phosphate, beta-tricalcium phosphate, calcium carbonate, barium carbonate, calcium sulfate, barium sulfate, polymorphs of calcium phosphate, sintered and non-sintered ceramic particles, and combinations of such materials. Additionally, “biocompatible inorganic materials” also include salts of di- or tri-valent metal cations such as copper, iron, manganese, and magnesium with anions such as, for example, chlorides and sulfates.

As used herein the term “collagen-based matrix” refers to extracellular matrices that comprise collagen fibers and include both naturally occurring extracellular matrices as well as reconstituted collagen matrices.

By “decreases” is meant a negative alteration of at least 10%, 25%, 50%, 75%, 100%, 200%, 300%, 400%, 500%, 1000%, or more.

As used herein the term “exogenous” or “exogenously added” designates the addition of a new component to a composition, or the supplementation of an existing component already present in the composition, using material from a source external to the composition.

As used herein an “effective” amount or a “therapeutically effective amount” of a composition refers to a nontoxic but sufficient amount of the composition to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

By “fragment” or “segment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increases” is meant a positive alteration of at least 10%, 25%, 50%, 75%, 100%, 200%, 300%, 400%, 500%, 1000%, or more.

The term, “parenteral” means not through the alimentary canal but by some other route such as subcutaneous, intramuscular, intraspinal, or intravenous.

As used herein the term “adipose stromal cells” refers to pluripotent stem cells recovered from adipose tissue. Typically the cells express at least one cell marker selected from the group CD14Oa, CD14Ob and NG2. Following contact with a chimeric polypeptide described herein the adipose stromal cell is differentiated to a chondrogenic lineage and has increased expression of type II collagen, Sox9, and Aggrecan relative to a control.

“Isolated” means altered or removed from the natural state. For example, a peptide or a nucleic acid naturally present in a living animal is not “isolated,” but the same a peptide or a nucleic acid partially or completely separated from the coexisting materials of its natural state is “isolated.” An isolated protein or nucleic acid can exist in substantially purified form, or can exist in a non-native environment such as, for example, a host cell.

As used herein, the term “purified” and like terms define an enrichment of a selected compound or selected cells relative to other components or cells normally associated with the selected compound or selected cells in a native environment. The term “purified” does not necessarily indicate that complete purity of the particular cells/compound has been achieved during the process. For example a purified adipose stromal cell comprises adipose stromal cells substantially free of adipocytes, endothelial cells and blood derived cells. Purified adipose stromal cells may be enriched 2, 3, 5, 10, 50, 100, 500, 1000-fold or more relative to other cell types (e.g., adipocytes, endothelial cells and blood derived cells).

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein a “linker” is a bond (e.g., covalent, ionic, or hydrogen bond), a molecule, or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups.

As used herein the term “patient” or “subject” without further designation is intended to encompass any warm blooded vertebrate domesticated animal (including for example, but not limited to livestock, horses, cats, dogs and other pets) and humans.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### BRIEF DESCRIPTION OF THE DRAWINGS

FIG. 1 includes three graphs. Chondrogenic differentiation of human adipose derived stem cells (hASCs) after treatment with several AB2 ligands. Real-time PCR analysis of selected chondrogenic markers (expression levels of type II collagen, aggrecan, and Sox9 mRNAs) after the cells were cultured for 14 days in complete chondrogenic medium containing the different chimeric ligands (Bone Morphogenic Proteins: BMP2 and BMP6 and AB chimeras AB235, AB204, AB208, AB211 and AB215). Gene expression is shown as fold change relative to cells cultured in incomplete chondrogenic medium (CTL). GAPDH expression was used for normalization. Data are shown as average ±SD (n=3) from at least 2 independent experiments.

FIGS. 2A-P includes micrographs. Shown are photographs of monolayer expanded cells cultured for 14 days with 10 ng/ml of different AB2 ligands and BMP2, showing a different increment of proteoglycans deposition (revealed by alcian blue labelling) depending on the ligands added to the medium (A). Higher resolution images of the same cells as in A are shown in B-K. L-O: Chondrogenic induction of the ligands was dose-dependent as demonstrated with the accumulation of toluidine blue positive material. Cells were cultured in chondrogenic medium with 10 ng/ml (L and N) or 50 ng/ml (M and O) of: AB235 (L-M) and TGF beta3 (N-O) and compared with control cells (P). Scale bars B-M: 6 mm

FIGS. 3A-T includes photographs and micrographs. Representative image of pellets (formed by centrifugation) in 15-ml conical tubes 6 weeks after culture in chondrogenic medium containing 10 ng/ml of different AB2 ligands (A-C); or BMP2 as positive control (D). Cells grown in incomplete chondrogenic medium were used as negative control (E). hASCs cultured in a pellet system (formed by detached monolayer) on day 42 treated with AB2 ligands (F-H); BMP2 (I) or non-treated cells (J). Toluidine blue stain of sections of the same pellets (K-O). Micrographs taken from these sections are provided (P-T).

FIG. 4A-D includes micrographs and graphs. Masson's Trichrome staining of AB235 treated cells in pellet cultures (A-C) showed marked collagen fibers with blue label. A high magnification view of (A) is shown in (B). A stained section from a different pellet is shown in (C), where the formation of collagen matrix lacunae can be seen. Expression of chondrogenic markers in a pellet culture (D). Pellets were cultured with AB235 or BMP2 for 6 weeks. RT-PCR analysis of chondrogenic gene markers (Col II, Col I, Sox9 and Aggrecan) normalized to GAPDH and shown as relative gene expression to cells cultured in incomplete chondrogenic medium. Data are shown as average ±SD (n=3) from at least 2 independent experiments. Original magnification: 5× for A; 40× for B and 20× for C.

FIG. 5A-L includes micrographs. Indirect immunofluorescence of monolayer cultured hASCs 4 weeks after treatment with AB204 (A-C), AB235 (D-F) and BMP2 (G-I) or grown in incomplete chondrogenic medium (J-L). Expression of cartilage-specific collagen II protein with intense green staining can be seen in treated cells, showing the characteristic collagen fibers framework of cartilage. 20× original magnification for: A, D, G and J; 40× original magnification for: B, C, E , F, H, I, K and L.

FIG. 6A-L includes micrographs. AB2 ligands-treated cell pellets formed a “cartilage like” tissue organization. hASC cell pellet cultures were grown in medium supplemented with AB235 (D-I) and incomplete chondrogenic medium (A-C) for 6 weeks, fixed and inmunostained with Collagen I (Col I) and Collagen II (Col II) antibodies. Protein expression was detected in red for Col I and green for Col II. Cell nuclei were labelled with DAPI (blue channel). Tissue organization in stratified layers showed Collagen I stained cells enveloping Collagen II producing cells Immunofluorescence of Sox9 was analysed after 6 weeks of treatment with AB235 (K-L) and control cells (J). Proteins were indirectly labelled with secondary antibodies (green channel) and cell nuclei labelled with DAPI (blue channel). Original magnification 20× for: A-F; 40× for: G-I; 63× for J-K and 100× for L.

FIG. 7A-D includes graphs. Gene expression analysis of hASC at different time-points during the differentiation protocol. hASC were cultured in AB235 and BMP2 enriched media for 2, 4, and 6 weeks. Total mRNA was obtained at each time-point, and transcript levels of Col II, Col I, Runx1 and Sox9 were analyzed by real-time PCR, normalized to GAPDH and shown as relative gene expression compared to cells cultured in incomplete chondrogenic medium. Data are shown as the average ±SD (n=3).

Table 1, which provides sequences, is provided at page 26 after the Examples.

### DETAILED DESCRIPTION

The invention features compositions comprising an adipose stromal cell having increased expression of type II collagen, Sox9, and Aggrecan, and methods of using such cells to treat cartilage injury or degeneration.

The invention is based, at least in part, on the discovery that Activin/BMP2 chimeric ligands (AB2 chimeras) created by mixing Activin and BMP-2 sequences (see Allendorph et al., PLoS One. 2011;6(11):e26402 and US published application no. 2010/0221777, the disclosure of which is expressly incorporated herein) are useful in promoting chondrogenesis. In short, BMP2 and Activin A sequences have been divided into 6 structural segments and these segments have been mixed to create the AB2 library of chimeras with novel functional properties. A systematic swapping strategy of the segments termed Random Assembly of Segmental Chimera and Heteromers (RASCH) was described in detail in Allendorph et al., 2011 (noted above). The chimeras are fully defined by the code (BXXXXX), where X=A (Activin A) or B (BMP2) depending on which segment is in position X. As shown herein AB2 chimeras (including for example, AB235) can efficiently (i) upregulate the expression of chondrogenic-related genes, (ii) induce collagens and proteoglycans synthesis and (iii) increase chondrogenic cell pellet size using human ASCs.

### Chondrocytes

Differentiated chondrocytes are capable of proliferating and secreting numerous growth factors and cytokines to form the extracellular matrix in mature cartilage. During chondrogenesis, these events are precisely regulated by different growth factors and soluble factors released from cartilage elements as well as from the perichondrium. Previous studies have shown that chondrocyte-derived factors may influence the fate of mesenchymal cells via paracrine, juxtacrine or gap-junction signalling pathways, suggesting that these factors can stimulate the formation of extracellular matrix in precursor cells and could be essential for regenerative medicine applications. These factors are, however, difficult to produce in sufficient quantity. The development of new molecules with increased activity in driving mesenchymal stem cells and adipose stem cells towards chondrogenic differentiation is, therefore, a priority in regenerative medicine and pharmaceutics.

A large quantity of BMP-2/BMP-6 heterodimer (BMP-2/6) can be produced by a chemical refolding method. BMP-2/6 is a better inducer of differentiation of human embryonic stem cells (hESCs) than its homodimeric counterparts (Valera et al., PLoS One. 2010;5(6):e11167). In addition, using a segment-swapping RASCH strategy with BMP-2 and Activin-A sequences, an AB2 library chimera, AB208, exhibits the refolding characteristics of BMP-2 while possessing Activin-like signaling attributes (Allendorph et al., PLoS One. 2011;6(11):e26402). These chimeras provide excellent candidates for stem cell differentiation/guidance.

In accordance with one embodiment compositions and methods are provided for stimulating the proliferation and/or differentiation of pluripotent cells into chondrocytes. In accordance with one embodiment a purified population of pluripotent stem cells is contacted with one or more AB2 chimeric polypeptides of Table 1 to induce proliferation and/or differentiation of the pluripotent stem cells to enhance the formation of chondrocytes. In one embodiment the pluripotent stem cells are recovered from adipose tissue and are contacted with a purified AB2 chimeric polypeptide selected from the group consisting of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab under conditions suitable for growth and/or proliferation of the pluripotent stem cells. In one embodiment adipose stromal cells are contacted with one or more purified AB2 chimeric polypeptides selected from the group consisting of 1b2a3b4b5a6a (AB204), 1b2a3b4b5b6a (AB215) and 1b2a3a4a5a6ab (AB235).

In a further embodiment the method comprises contacting a purified population of adipose stromal cells with the AB235 polypeptide having the sequence M Q A K H K Q R K R L K S S C K R H P L Y V D F S D V G W N D W I I A P S G Y H A N Y C E G E C P S H I A G T S G S S L S F H S T L V N H Y R M R G H S P F A N L K S C C V P T K L R P M S M L Y Y D D G Q N V V L K N Y Q D M I V E E C G C S. In one embodiment the purified population of adipose stromal cells comprises a population of cells enriched for adipose stromal cells relative to other cells present in the tissue from which said adipose stromal cells were purified. In one embodiment the purified adipose stromal cells are substantially free from adipocytes, endothelial cells and blood derived cells. For example the purified adipose stromal cells population contains less than 5%, 2%, 1% or 0.1% of total adipocytes, endothelial cells and blood derived cells relative to the total cell population.

In accordance with one embodiment adipose stromal cells are induced to express chondrogenic-related genes, and thus differentiate into chondrocytes, by contacting the cells with a purified AB2 chimeric polypeptide selected from the group consisting of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab to produce a mixture, wherein the mixture is incubated under conditions conducive for the proliferation and/or growth of said cells, resulting in the enhanced expression of chondrogenic-related genes. In one embodiment the upregulated chondrogenic-related genes are selected from the group consisting of collagen II, aggrecan and Sox9 expression.

In one embodiment the purified adipose stromal cells are contacted with one or more of the purified AB2 chimeric polypeptides disclosed herein and the mixture is incubated in vitro using standard culture media to expand and differentiate the cells into chondrocytes. In an alternative embodiment the purified adipose stromal cells are contacted with one or more of the purified AB2 chimeric polypeptides disclosed herein and the mixture is implanted or injected into a warm blooded vertebrate with or without first incubating the cell in vitro. In one embodiment the purified adipose stromal cells are isolated from the same patient that the cells are injected after exposure to the AB2 chimeric polypeptide (i.e., the adipose stromal cells represent a native autologous cell populations relative to said warm blooded vertebrate). In one embodiment wherein the cells are implanted or injected into a patient, the mixture is injected into said vertebrate at the site where enhanced chondrocytes formation is desired.

In one embodiment a composition comprising purified adipose stromal cells and one or more of the purified AB2 chimeric polypeptides is provided wherein the composition further comprises an extracellular matrix protein or glycoprotein or a biocompatible polymer selected from the group consisting of collagen, fibronectin, polyglycol acid (PGA), polylactic acid (PLA) and a co-polymer of PGA and PLA. In one embodiment a composition is provided comprising purified adipose stromal cells, one or more AB2 chimeric polypeptides and a biocompatible polymer, wherein the AB2 chimeric polypeptides are linked, preferably covalently bound to, to the biocompatible polymer. In a further embodiment the adipose stromal cells are encapsulated or entrapped within a biocompatible polymer matrix, optionally with one or more AB2 chimeric polypeptides linked to the polymer matrix. In a further embodiment the AB2 chimeric polypeptides comprising compositions disclosed herein may further comprise a pharmaceutically acceptable carrier. In one embodiment the composition comprises an AB2 chimeric polypeptide having the sequence MQAKHKQRKRLKSSCKRHPLYVDFSDVGWNDWIIAPSGYHANYCEGECPSHIAGTS GSSLSFHSTLVNHYRMRGHSPFANLKSCCVPTKLRPMSMLYYDDGQNVVLKNYQD MIVEECGCS. In one embodiment a composition is provided comprising an AB2 chimeric polypeptide linked to a biocompatible polymer wherein the AB2 chimeric polypeptide comprises the sequence MQAKHKQRKRLKSSCKRHPLYVDFSDVGWNDWIIAPSGYHANYCEGECPSHIAGTS GSSLSFHSTLVNHYRMRGHSPFANLKSCCVPTKLRPMSMLYYDDGQNVVLKNYQD MIVEECGCS.

In accordance with one embodiment a composition is provided for stimulating the proliferation and/or differentiation of pluripotent cell into chondrocytes. In one embodiment the composition comprises a mixture of one or more AB2 chimeric polypeptides selected from Table 1 and a suitable culture media for growing cells in vitro. In one embodiment the composition comprises an AB2 chimeric peptide having the sequence of MQAKHKQRKRLKSSCKRHPLYVDFSDVGWNDWIIAPSGYHANYCEGECPSHIAGTS GSSLSFHSTLVNHYRMRGHSPFANLKSCCVPTKLRPMSMLYYDDGQNVVLKNYQD MIVEECGCS; and culture media comprising Dulbecco's modified Eagle's medium with 10% fetal bovine serum.

In one embodiment a kit is provided for inducing the differentiation of pluripotent cells to chondrocytes wherein the kit comprises a purified AB2 chimeric polypeptide and culture media and/or culture labware. In one embodiment a kit for inducing the formation of chondrocytes form adipose stromal cells is provided wherein the kit comprises a purified chimeric polypeptide selected from the group consisting of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab, a suitable cell culture media, and instructional material. In one embodiment the kit comprises a chimeric peptide having the sequence of MQAKHKQRKRLKSSCKRHPLYVDFSDVGWNDWIIAPSGYHANY CEGECPSHIAGTSGSSLSFHSTLVNHYRMRGHSPFANLKSCCVPTKLRPMSMLYYDD GQNVVLKNYQDMIVEECGCS; and the culture media comprising Dulbecco's modified Eagle's medium with 10% fetal bovine serum. In a further embodiment the kit further comprises a biocompatible polymer, including for example a biocompatible polymer selected from the group consisting of collagen, fibronectin, polyglycol acid (PGA), polylactic acid (PLA) and a co-polymer of PGA and PLA. In a further embodiment the kit also comprises a purified population of adipose stromal cells.

As disclosed herein the chondrogenic potential of A/B2 chimera ligands on hASC was investigated. AB2-ligands have been found to increase collagen II, aggrecan or Sox9 expression, thus demonstrating a potential in inducing chondrogenic differentiation. The level of induction of chondrogenic genes was highly dependent on the chimeric ligands used, with AB235 showing the strongest chondrogenic potential both in monolayer and pellet culture systems. Time point evaluations confirmed that chondrogenic differentiation induced by AB235 exhibited a similar pattern to that of the cartilage maturation process with an initial increase of Col I and Sox9 and a decline at later times, a time-dependent increase in expression of Col II, and a marked decrease of Runx1 levels. Sox9 acts in early stages of chondrocyte differentiation, which directly induces type II collagen and is expressed in mesenchymal condensations. Decreased Col I gene expression suggested that AB2 ligands induced articular chondrocyte differentiation, as type-I collagen is either present in very small amounts or absent in hyaline cartilage, but abundant in fibrocartilage.

Runx1 is a transcription factor highly expressed during early chondrogenesis and is downregulated in late stages of chondrocyte differentiation. Moreover, it has been demonstrated that Runx1 is capable of accelerating induction of MSC differentiation towards chondrogenesis by increasing the expression of early chondrocyte maturation markers. Thus, the results indicate that AB235 is superior in inducing chondrogenesis from early to late stages of maturation.

Members of the TGF-β superfamily, TGF-β1 or TGF-β3, have been shown to induce chondrogenic differentiation. The TGF-β family is divided into two general branches, whose members have diverse but often complementary effects: the TGF-β/Activin/Nodal branch and the BMP/GDF branch. TGF-β molecules signal through a heteromeric cell surface serine/theorine kinase receptor complex consisting of a dimeric ligand and a pair of both type I and type II receptors. The type I receptors are termed activin receptor-like kinases (ALKs). The TGF-β ligands signal upon binding to one of five type II TGF-β receptor kinases and subsequent cross-phosphorylation of one of seven type I receptors which triggers phosphorylation of the cytoplasmic receptor-regulated SMAD molecules. BMPs have a synergistic effect with TGF-β on promoting MSCs into hyaline-like cartilage tissue and also signal through activin type II receptors (ActrII) or ALKs. In addition, signaling through the BMP receptors is required for the maintenance of the articular cartilage in post-natal organisms.

The results reported herein showed that AB235 enhanced hASCs chondrogenic differentiation in comparison with individual treatment with BMP2, suggesting that this chimeric ligand could signal through both type I and type II receptors more efficiently, thereby increasing cartilage maturation. In fact, both BMP2 and Activin can bind ActrII (activin with very high affinity and BMP with lower affinity). Moreover, differences in the level of endogenously secreted growth factors and/or in the expression of their specific receptors might modulate the resulting phenotype of the target cell and could be responsible for the success of chondrogenic differentiation in MSC.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Material and Methods

Generation of TGF-β Chimeras

The AB2 library was generated as previously described (Allendorph et al. PLoS One. 2011;6(11):e26402) and in U.S. patent application Ser. No. 12/712,068, which is incorporated by reference in its entirety. Briefly, the mature domains of both human BMP2 and human Activin A were divided into 6 segments and primers were designed for each segment on the basis of their structural elements and the sequence similarity between them.

The crystal structures of BMP2 and human Activin A were compared and 6 distinct structural segments were identified to minimize disruption of secondary structural elements thus to conserve local structural elements when recombining the segments. The exact segment boundaries were further refined following a protein sequence alignment of these ligands with additional adjustment of segmental boundaries to allow amino acids conserved for segment swapping universally (universal joints) among the entire TGFβ superfamily ligands as seamlessly as possible. Sequence alignment of BMP2 and human Activin A polypeptide sequences, as well as those of the 6 segments is provided below.

| | | | |
|---|---|---|---|
| BMP2 | 1 | QAKHKQRKRLKSSCKRHPLYVDFSDVGWNDWIVAPPGYHAFYCHGECPFP | 50 |
| Activin A | 1 | --GLECDGKVNICCKKQ-FFVSFKDIGWNDWIIAPSGYHANYCEGECPSH | 47 |
| BMP2 | 51 | LADHLNSTNHAIVQTLVN----SVNSKIP--KACCVPTELSAISMLYLD | 93 |
| Activin A | 48 | IAGTSGSS-LSFHSTVINHYRMRGHSPFANLKSCCVPTKLRPMSMLYYD | 95 |
| BMP2 | 94 | ENEKVVLKNYQDMVVEGCGCR | 114 |
| Activin A | 96 | DGQNIIKKDIQNMIVEECGCS | 116 |
| Segment 1 | | | |
| BMP2 | 1 | QAKHKQRKRLKSSCKRHPLYVDFSDVGWND | 30 |
| Activin A | 1 | --GLECDGKVNICCKKQ-FFVSFKDIGWND | 27 |
| Segment 2 | | | |
| BMP2 | 31 | WIVAPPGYHAFYCHGECP | 48 |
| Activin A | 28 | WIIAPSGYHANYCEGECP | 45 |
| Segment 3 | | | |
| BMP2 | 49 | FPLADHLNSTNHAIVQTLVN | 68 |
| Activin A | 46 | SHIAGTSGSS-LSFHSTVIN | 64 |
| Segment 4 | | | |
| BMP2 | 69 | ----SVNSKIP--KACCVP | 82 |
| Activin A | 65 | HYRMRGHSPFANLKSCCVP | 84 |
| Segment 5 | | | |
| BMP2 | 83 | TELSAISMLYLD | 94 |
| Activin A | 85 | TKLRPMSMLYYD | 96 |
| Segment 6 | | | |
| BMP2 | 95 | WIVAPPGYHAFYCHGECP | 114 |
| Activin A | 97 | WIIAPSGYHANYCEGECP | 116 |

Specifically, the pre-helix loop and the majority of the a-helix were combined into Segment 3, while the remainder of the a-helix and the beginning of beta strand 3 were placed into Segment 4. A few additional point mutations were necessary to create universal joints between certain segments. At the end of Segment 3, TLVN of BMP2, which corresponds to TVIN of the Activin A sequence, was taken as the consensus sequence. At the end of Segment 5, LYLD of BMP2 is equivalent to LYYD of Activin A, for which LYYD was taken as the consensus sequence. Finally, the N-terminus of Activin-βA contains 2 additional cysteines, forming a 4th intra-disulfide bond, compared to BMP2. To avoid the potential complication in the refolding process by these extra disulfide bond, Segment 1 of Activin A was removed from the final pool of segments. Thus, the pool consisted of 11 Segments in total (Segments 1 through 6 of BMP2 and Segments 2 through 6 of Activin A).

An overlapping PCR strategy was used to mix the various segments together to generate full-length PCR fragments of each chimera. For the Activin A/BMP2 chimeras, the mature domains of human BMP2 and human Activin A were initially divided into 6 sections each and primers were designed for each section. For BMP2, the primers coded for the following protein sequences:

Section 1, QAKHKQRKRLKSSCKRHPLYVDFSDVGWND;
Section 2, WIVAPPGYHAFYCHGECP;
Section 3, FPLADHLNSTNHAIVQTLVN;
Section 4, SVNSKIPKACCVP;
Section 5, TELSAISMLYYD;
Section 6, ENEKVVLKNYQDMVVEGCGCR.

For Activin A, the primers coded for the following protein sequences:

Section 1, RGLECDGKVNICCKKQFFVSFKDIGWNDW;
Section 2, WIIAPSGYHANYCEGECP;
Section 3, SHIAGTSGSSLSFHSTLVN;
Section 4, HYRMRGHSPFANLKSCCVP;
Section 5, TKLRPMSMLYYD;
Section 6, DGQNIIKKDIQNMIVEECGCS.

To generate the 1b chimeras, two oligos were used to insert the BMP2 sequence QAKHKQRKRLKSSCKRHPLYVDFSDVGWNDII into the target gene. Outer primers for all constructs were constructed to incorporate a 5′ NdeI site and a 3′ XhoI site for cloning into pET21a expression vector. The AB235 chimera was created from AB208 (BAAAAA) by mutating IIKKDIQN from segment 6 to VVLKNYQD using a Quickchange kit. The chimera 1b2a3a4a5a6a L66V/V67I (AB2-009) was created from AB2-008 by introducing a valine and iso-leucine at the indicated positions (denoted as “L66V/V67I”). The chimera 1b(1a_II)2a3a4a5a6a (AB2-010) was created from AB2-008 by introducing a segment KKQ-FFVSFKDI (denoted as “(1a_II)”), which replaces the second half of the 1b section with the corresponding sequence from Activin A. The desired protein sequences were confirmed by DNA sequencing. Proteins were expressed in E. coli as inclusion bodies and purified as previously described (Allendorph et al.).

The chimeras were labeled according to the sections they contained. For example, 1b2b3b4a5a6b, in which the b's represent that the section was taken from BMP2 and the a's represent that the section was derived from Activin A. The chimeras were also given shorthand numeric designations, such as A/B2-020, so that any functional assays could be undertaken in a blind manner. Table 1 sets forth the various chimeras.

ASCs Cell Culture

Human mesenchymal stem cells were derived from the adipose tissue from the subcutaneous abdomen of a 37-year-old Caucasian female (lot number 9061601.12, PromoCell, Heidelberg, Germany). Cells were cultured in “growing medium”: high glucose Dulbecco's modified Eagle's medium (DMEM, Invitrogen) with 10% fetal bovine serum and 1% penicillin/streptomycin (Invitrogen).

Induction of Monolayer Expanded Cells Towards Chondrocytes

hASCs were induced to chondrogenic phenotype as previously described (Estes et al. Nat Protoc. July 2010;5(7):1294-1311) with slight variation. Briefly, cells were seeded at 30-40% confluence in a 12 or 24-well plate. Non-treated cells or control cells were grown in either growing medium (CTL1, see above) or in “incomplete chondrogenic medium” (CTL2): DMEM-high glucose (Invitrogen) supplemented with 10% fetal bovine serum and 1% ITS+Premix (Collaborative Biomedical-Becton Dickinson, Bedford, Mass.), in the presence of 1% penicillin-streptomycin (Invitrogen). In addition, 50 μg/μL of 1-ascorbic acid 2-phosphate (Sigma-Aldrich) was added fresh during each media exchange. To direct chondrogenic differentiation, cells were cultured in “chondrogenic medium”: incomplete chondrogenic medium containing 10 ng/ml of the chimeric ligand. Media was changed every other day. Cells were cultured in chondrogenic media for 2, 4 and 6 weeks depending on the experiment. Complete chondrogenic medium with 10 ng/ml of BMP2, known to be capable of chondrogenesis, was used as positive control. In addition, the chondrogenesis potential of AB2 chimera ligands and TGF beta3 was compared at two different concentrations: 10 ng/ml and 50 ng/ml.

Chondrogenic Differentiation in Cell Pellet Culture

Two different methods were used to induce the formation of a cell pellet culture. In the first method, a suspension of 250,000 cells, in either growing media, incomplete chondrogenic medium or chondrogenic medium, was added to 15-ml conical tubes and centrifuged at 300 g at 21° C. for 5 min to form a pellet at the bottom of each tube. The pellets appeared as a round-shaped mass at the bottom of the tube. In the second method, cells were grown in 12-well plates in chondrogenic medium and after confluency, the monolayer spontaneously detached from the plastic and took the form of a crumpled paper ball. Control cells grown in incomplete chondrogenic medium did not detach spontaneously from the plastic and, therefore, the monolayer was manually separated using a sterile tip. The emerging pellets were carefully transferred to 15-ml conical tubes. Tubes were incubated with loosened tops at 37° C. and 5% CO₂. Medium was exchanged every other day for the duration of the experiment, and tubes were gently shaken to avoid the adherence of the pellet to the plastic walls.

RNA Isolation and Real Time-PCR Analysis

Real-time PCR was performed to study the differentiation profile of the hASCs in order to analyze the expression of key cartilage markers. Total cellular RNA was isolated using Trizol Reagent (Invitrogen) according to the manufacturer's recommendations. 2 μgs of DNAse1 (Invitrogen) treated total RNA was used for cDNA synthesis using the SuperScript II Reverse Transcriptase kit for RT-PCR (Invitrogen). Real-time PCR was performed using the SYBR-Green PCR Master mix (Applied Biosystems). Sequences of primers were 5′GGACTCATGACCACAGTCCATGCC3′ and 5′TCAGGGATGACCTTGCCCACAG3′ for GAPDH; 5′AGGATGGCTTCCACCAGTGC3′ and 5′TGCGTAAAAGACCTCACCCTCC3′ for aggrecan (ACN); 5′GAGACAGCATGACGCCGAG3′ and 5′ GCGGATGCTCTCAATCTGGT3′ for type II collagen (COL2A1); 5′ATGGATGAGGAAACTGGCAACT3′ and 5′GCCATCGACAAGAACAGTGTAAGT3′ for type I collagen (COL1A1); 5′ ACTCCGAGACGTGGACATC3′ and 5′TGTAGGTGACCTGGCCGTG3′ for Sox9 (SOX9); 5′AGAACCTCGAAGACATCGGC3′ and 5′GGCTGAGGGTTAAAGGCAGTG3′ for Runx1. The gene expression levels were normalized to corresponding GAPDH values and are shown as fold change relative to the value of the control sample. All the samples were done in triplicate for each gene.

Cell Monolayer and Cell Pellet Processing

For histological and immunocytochemistry analyses, monolayer expanded cells and culture pellets were processed as follows. Culture pellets were fixed with 4% paraformaldehyde for 20 min at room temperature and embedded in 2.3 M sucrose for 1 hour. Cell pellets were embedded in Tissue Freezing Medium, Blue (Electron Microscopy Sciences) and frozen on dry ice. Sections of 2-6 μm in thickness were cut with a microtome and placed in the centre of a coated slide. Sections were washed with PBS in a humid chamber, until the excess sucrose washed away. Monolayer expanded cells were washed thrice in PBS and fixed with 4% paraformaldehyde for 20 min at room temperature (RT).

Histochemical Determinations

Toluidine blue staining: 0.1 gram of toluidine blue (Sigma) was dissolved in 100 ml of dH2O. Fixed cells and pellet sections were stained in toluidine blue solution for 1-5 min at RT, and rinsed with dH2O until the excess stain washed away. Alcian Blue Staining: 1 gr of Alcian blue (Sigma) was diluted in 3% acetic acid solution, and pH was adjusted to 2.5 with acetic acid. Fixed cells and pellet sections were stained in Alcian blue solution for 20 min at RT, and washed with dH2O until the dye was gone. Masson's Trichrome Staining: we used Masson's trichrome kit (Sigma) for the detection of total collagen content. The collagen fibers will be stained blue, the nuclei will be stained dark brown/purple and the cytoplasm is stained red/pink.

Fluorescence Microscopy

Briefly, after fixation, cells and sections were blocked and permeabilized for 1 hr at 37° C. with 5% BSA/5% appropriate serum/1×PBS with 0.1% Triton X100. Subsequently, cells and sections were incubated with the indicated primary antibody overnight at 4° C. The cells and sections were next washed thrice with 1×PBS and incubated for 2 hr at 37° C. with the respective secondary antibodies and washed thrice with 1×PBS. DAPI (0.5 μg/ml in PBS) was used to visualize the nuclei. Cells were mounted with aqueous mounting media before analysis. Primary antibodies used were anti-type I collagen antibody (rabbit polyclonal antibody (ab292), Abcam, Cambridge, Mass.), anti-type II collagen antibody (mouse monoclonal antibody (ab3092), Abcam, Cambridge, Mass.), type X collagen (Neomarkers, Fremont, Calif.) or anti-Sox9 antibody (rabbit polyclonal antibody (AB5535), Chemicon). Secondary antibodies were Alexa Fluor 568 or 488 (Neomarkers). The pictures were taken with either a Leica TCS SP2 AOBS confocal or a Nikon E-800 microscope.

### Results

Upregulated Expression of Chondrogenic Genes after Induction with Chimeric Ligands in ASCs

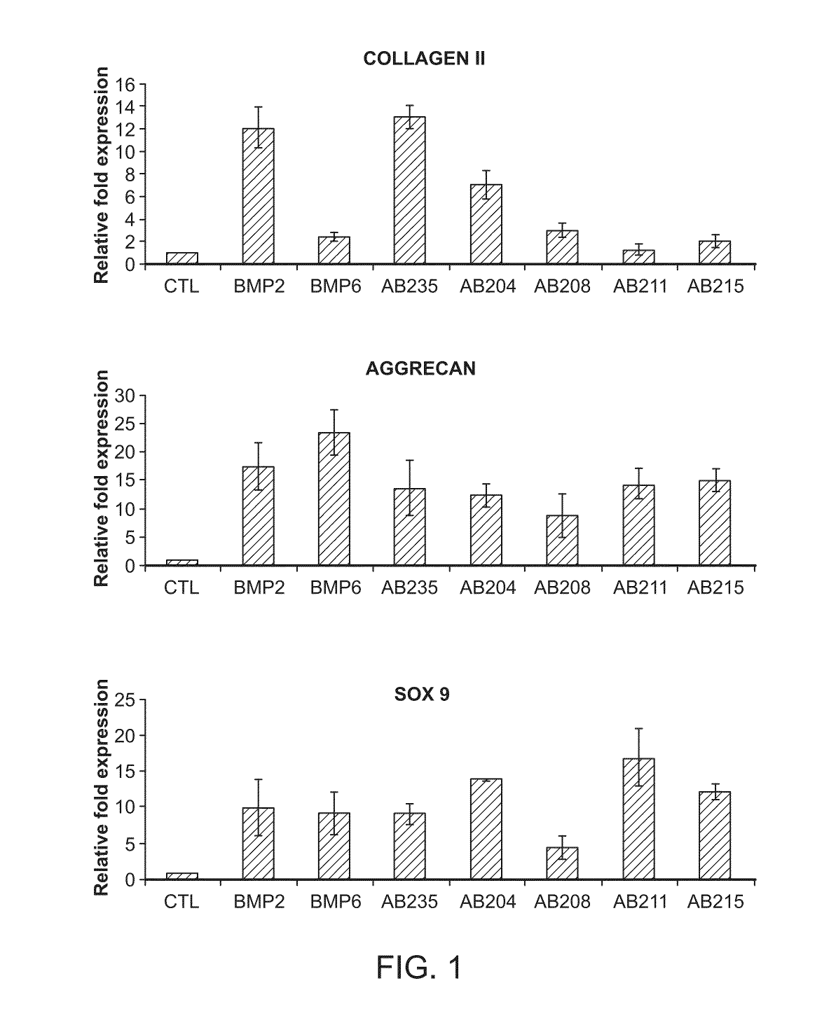

To select chimeric ligands with higher ability to induce chondrogenesis, real time PCR analyses of cells grown in monolayer for four weeks in chondrogenic induction media was performed. Enhanced gene expression of the principal cartilage extracellular matrix components, type II collagen (Col II) and aggrecan was observed in all of the chimeras tested when compared to control cells, suggesting the enormous potential of these constructs (FIG. 1). Among all the chimeras, AB235 showed the most prominent chondrogenic effect since it markedly upregulated Col II mRNA to an average of 13-fold compared with the control. This chimera also increased aggrecan mRNA expression 14-fold and Sox 9 mRNA expression 9-fold. In addition, AB204 and AB215 also showed increased expression of these chondrogenic markers (FIG. 1). Further studies were completed to fully assess the chondrogenic induction capacity of these three AB2 ligands: AB235, AB204, and AB215.

Formation of Extracellular Proteoglycan Matrix

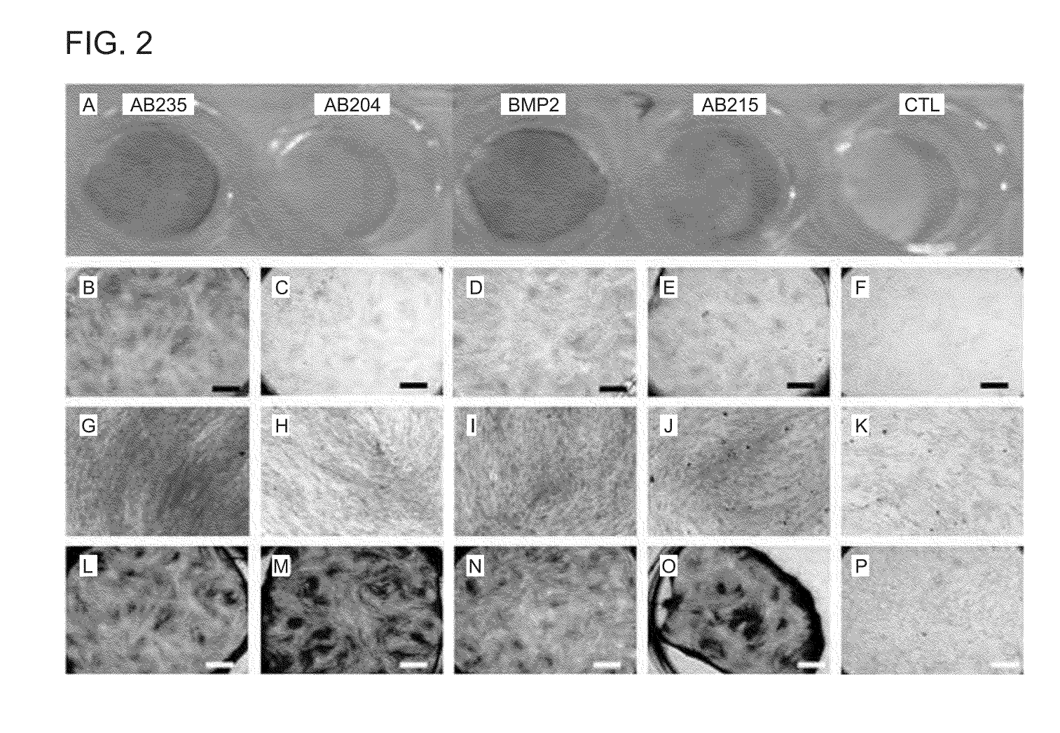

The degree of maturation after chondrogenic differentiation was further assessed by toluidine and Alcian blue staining of monolayer cultured cells. Accumulation of glycosaminoglycans in the extracellular environment indicates the formation of a cartilage matrix, and can be positively stained by toluidine and Alcian blue. A noticeable increase in the intensity of Alcian blue staining in cells treated with AB235 for two weeks was observed when compared with control cells (CTL) or cells treated with different chimeras, such as AB204 and AB215. Furthermore, the intensity of the Alcian blue staining was comparable between AB235- and BMP2-treated cells (positive control cells) (FIG. 2A-K). FIG. 2G-K shows, in more detail, the striated distribution of the cartilage extracellular matrix and a strong blue staining for AB325- and BMP2-treated cells is apparent (FIG. 2G and I, respectively). Toluidine blue staining was carried out on in cells treated with two different concentrations of AB235 (FIG. 2L-M) and TGFbeta3 (FIG. 2N-O). AB235- and TGF-β3-treated cells were observed to showed a similar dose-dependent increase in the intensity of toluidine blue staining. However, the intensity of staining in control cells was low (FIG. 2P). These results revealed the presence of acidic proteoglycans characteristic of the cartilaginous matrix in cells treated with chimeric ligands.

Formation of Cartilage-Like Tissue

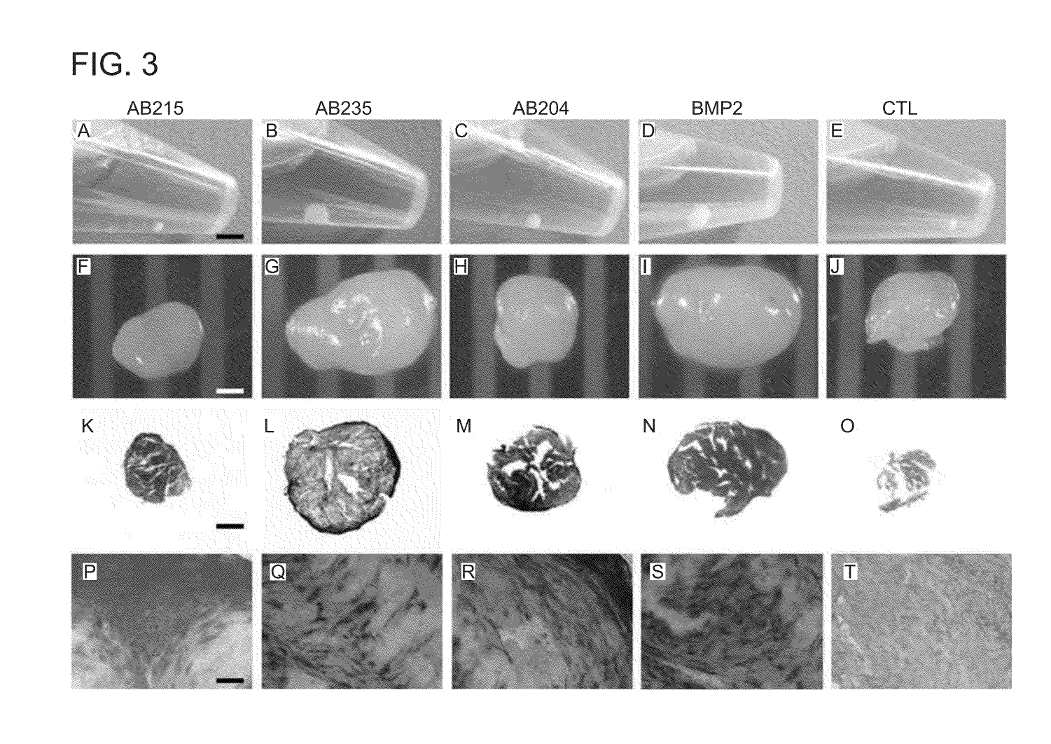

A cell pellet culture model which requires a high cell density was used to create compact cell-cell contacts mimicking the cellular condensation process occurring in normal limb development (Johnstone et al. Exp Cell Res. Jan. 10, 1998; 238(1):265-272). The method to induce pellet formation as a detached monolayer with “crumpled paper ball” form has not been used before to induce chondrogenesis). FIG. 3A-J shows that the cell pellets achieved a cartilage-like appearance with a white shiny look. In addition, both pellets prepared with the traditional centrifugation method (FIG. 3A-E) and with the new methodology (FIG. 3F-J) differed in size depending on the ligands included in the medium. In both methods, a significant increase in cell pellet size was observed when AB235 or BMP2 was added to the medium (FIGS. 3B and G) in comparison with cell pellets grown in incomplete chondrogenic medium with no ligands (FIGS. 3E and J), or with AB215 (FIGS. 3A and F), or AB204 (FIGS. 3C and H). Furthermore, a pellet could not be formed when cells were fed only with growing medium (data not shown). The formation of cartilage-like tissue was investigated using toluidine blue staining in cell pellet sections. Active matrix production was detected in AB2 chimeras- and BMP2-supplemented pellet cultures (FIG. 3K-N). Note that the control cells (FIG. 30) did not display tissue-like organization and rather present the appearance of a cell aggregation with no clear organization. A non-uniform distribution pattern of toluidine blue staining in treated cells is shown in more detail in FIG. 3P-T. These dense regions of a characteristic positive metachromatic staining are evidence of glycosaminoglycans synthesis (FIG. 3P-T).

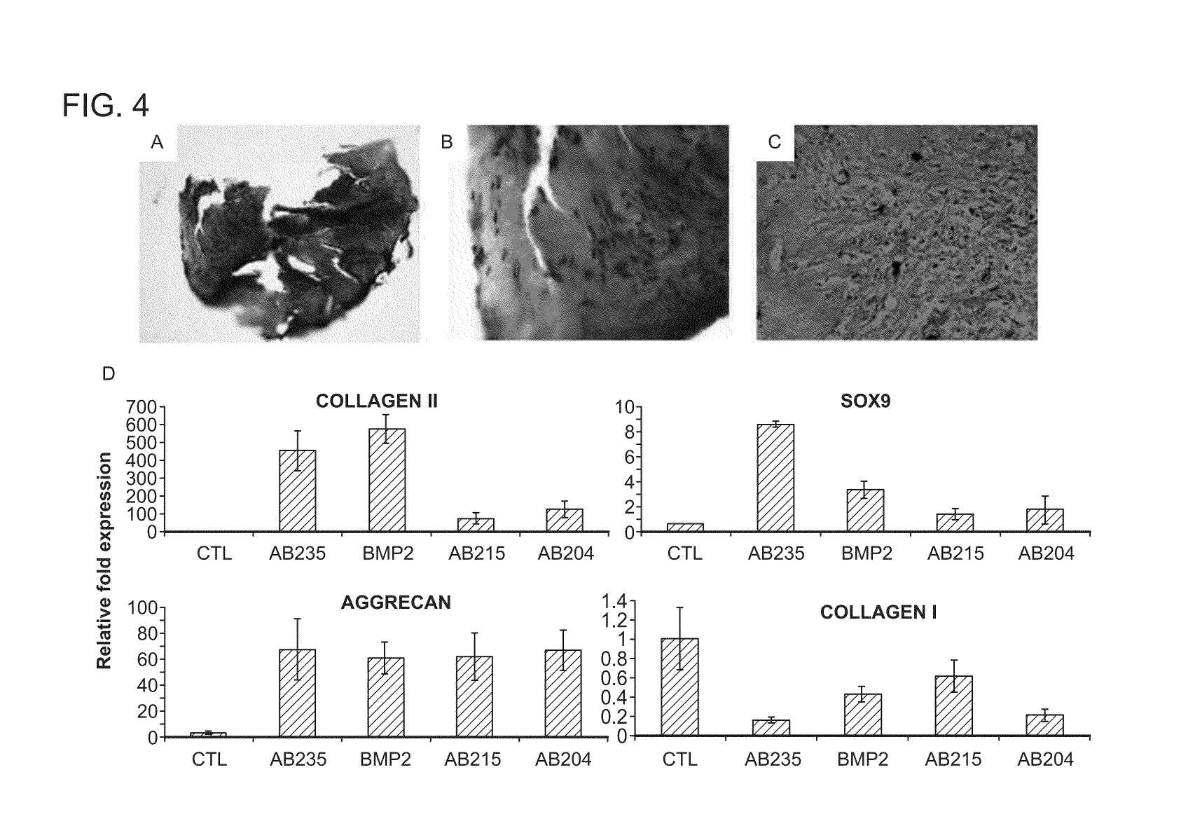

Furthermore, the Masson's trichrome staining of pellet cultures grown with AB235 supplementation revealed the typical appearance of cartilage tissue with blue staining for collagen fibber deposits that form a dense matrix (FIG. 4A-B) containing the differentiated chondrocytes, stained in dark brown, within lacunae (FIG. 4C). These results reinforce the observed high potential of AB235 in promoting chondrogenesis.

To quantify the chondrogenic potential of the hASC cultured in a pellet system, real-time PCR analysis was performed of genes related to cartilage differentiation 6 weeks after treatment with chimeric ligands (FIG. 4D). Upon AB235 treatment, increased expression of a number of chondrogenic markers was observed including type II collagen (79 fold), Sox9 (14 fold) and Aggrecan (20-fold) as compared with the control, whereas the expression of the fibrous tissue marker type I collagen (Col I) decreased. These results indicated that ASCs were successfully induced towards an articular chondrogenic lineage by the chimeric ligand AB235.

Chondrogenic Protein Expression

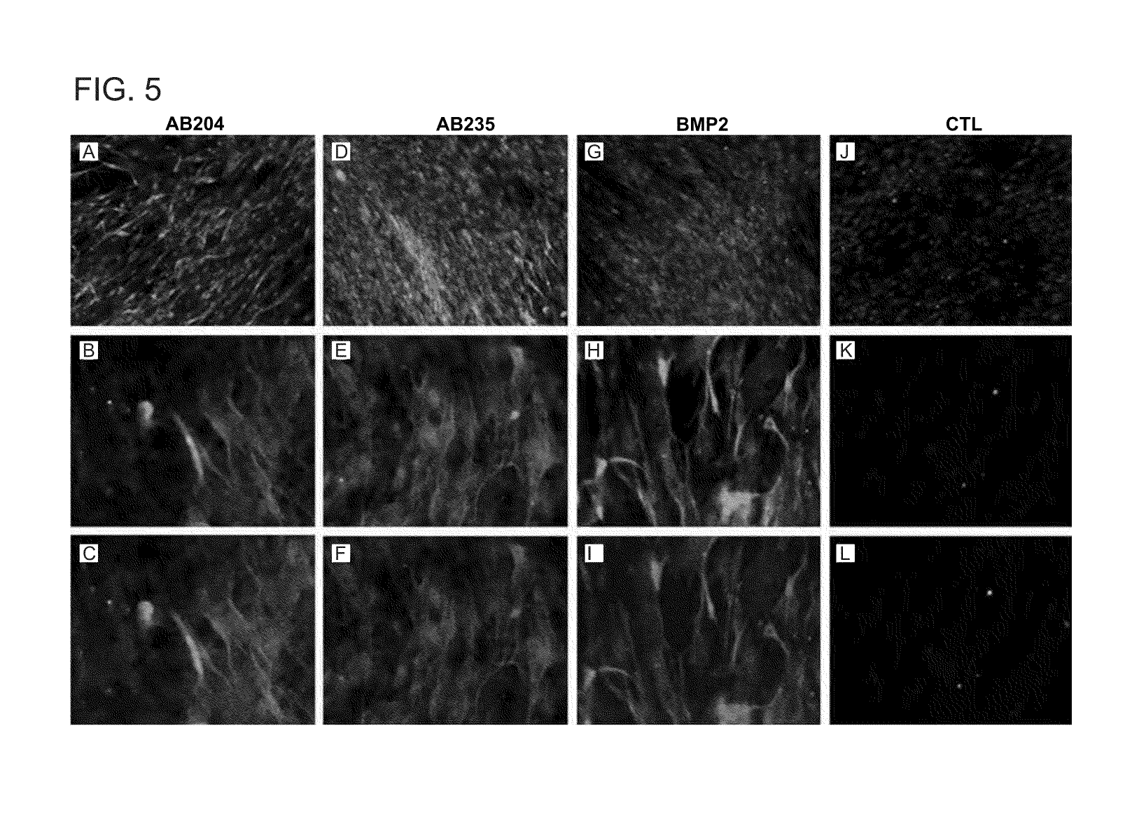

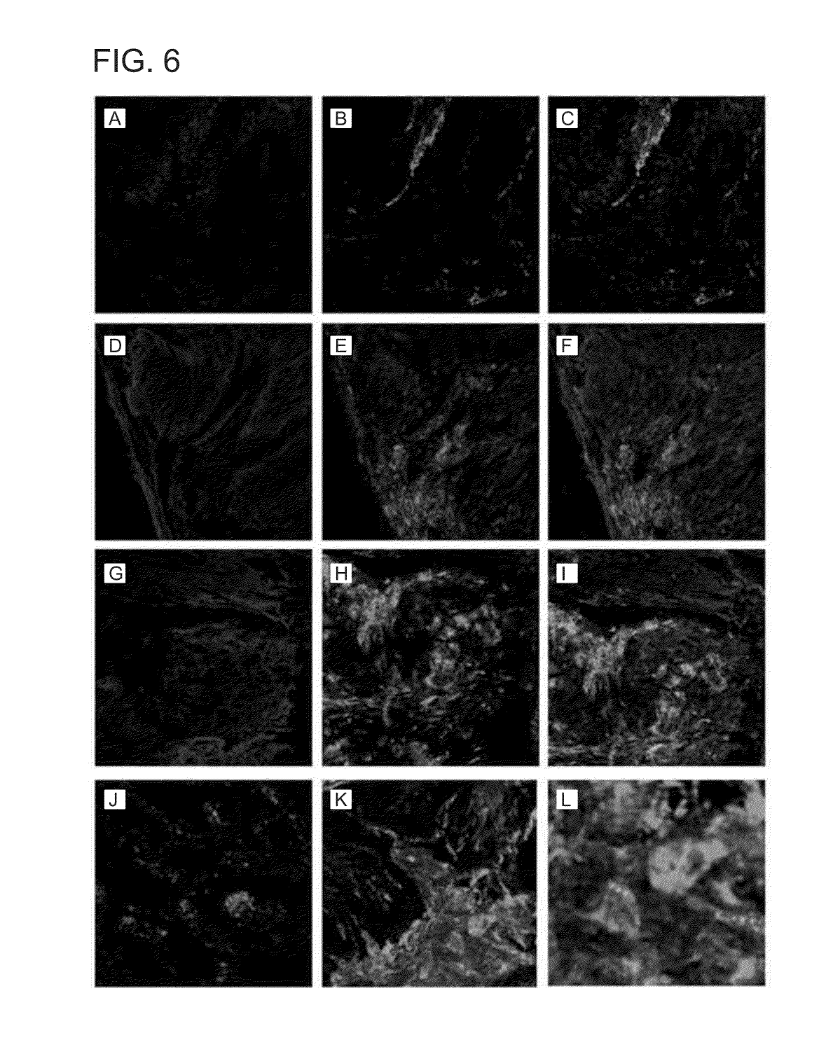

To further evaluate the chondrogenic potential of the chimeric ligands, immunocytochemistry assays was performed on cells grown in monolayer for 4 weeks (FIG. 5A-L) or a pellet system differentiated after 6 weeks (FIG. 6A-L). Monolayer cultured cells treated with AB204 (FIG. 4A-C) and AB235 (FIG. 4D-F) clearly demonstrated an increase in the expression of type II collagen which develops a dense filamentous matrix network deposited over the cells (FIG. 4B-C FIG. 4E-F respectively). Furthermore, immunostaining of Col II and Col I in AB235-treated cells cultured in a pellet system (FIG. 6) also showed a substantial number of Col II positive cells (FIGS. 6E, F, H and I), which formed a dense filamentous network of collagen II connecting the cells. Interestingly, we did not detect co-localization of collagen I and II as Col I-positive cells were located surrounding Col II-positive cells, which strongly resemble a stratified structure in the tissue (FIG. 6F and I). Control cells (FIG. 6A-C) did not show tissue-like appearance or a strong, positive label of the chondrocyte markers tested.

In addition, expression of the chondrogenic transcription factor Sox9 was highly enriched in AB253-treated cells (FIG. 6Q-L) when compared with control cells (FIG. 6J) in pellet sections. Finally, the pellet section was stained with type X collagen, a marker of hypertrophic chondrocytes committed to the osteogenic lineage, and did not observe significant positive staining. Altogether, these data suggested that ASCs acquired a mature chodrocyte-like phenotype induced by the activity of the chimeric ligand AB235.

Time Points of Chondrogenesis Induction

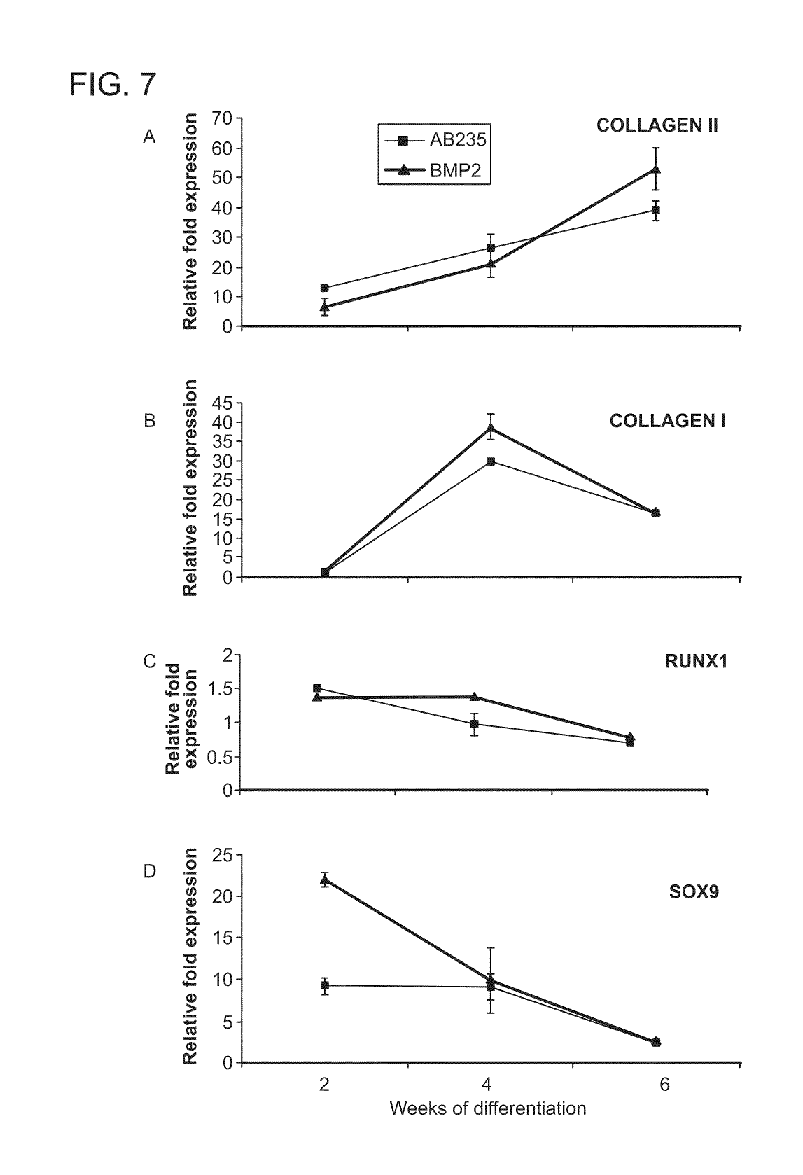

To analyze the timing of the chondrogenic differentiation process during the exposure of the hASC to chimeric ligands, samples were collected at different time points (2, 4 and 6 weeks) and the expression of some early (Sox9 and Runx1) and late (Col II and I) markers of the chondrogenic pathway were studied. FIG. 7A shows that Col II gene expression increased over the period of differentiation and reached a 39-fold increment 6 weeks after the treatment with AB253. Col I expression was significantly increased in both treatment groups (AB235 and BMP2) as compared to control cells (FIG. 7B) reaching maximum expression after four weeks of treatment. Moreover, after 21 days of treatment with chondrogenic medium we observed a modest and transient enhancement of the Runx1 mRNA level, which was more evident in AB235 treated cells. The Runx1 expression level decreased in later time-points for all treatments (FIG. 7C). Chondrocyte differentiation is initiated by expression of the transcription factor Sox9. After 2 weeks of culture in media supplemented with BMP2, Sox9 expression enhanced 21-fold but dropped to 9-fold after 4 weeks. The addition of AB235 to the media also increased Sox9 gene expression 9-fold after four weeks of culture. After 6 weeks of culture the expression of this transcription factor decreased for both treatments (FIG. 7D).

The data provided herein indicate the present invention represents an optimized, reproducible and inexpensive protocol to direct the differentiation of adipose stem cells into the chondrogenic lineage. For example, on specific chimeric ligand AB235 has proven to be highly efficient to induce chondrogenesis from hASC. This novel differentiation culture system has broader clinical applications.

Table 1 is provided below.

TABLE 1

| BMP-2/activin | Sample | | | |
|---|---|---|---|---|
| constructs | Designation | DNA Sequence | Protein Sequence | Exemplary Characteristics |
| 1b2b3a4a5a6a | AB2-001 | ATGCAAGCCAAACACAAACAGCGGAAACGCCTTAA | M Q A K H K Q R K R L E S S C K R H P L Y V D F S D | Potential universal |
| | | GTCCAGCTGTAAGAGACACCCTTTGTACGTGGACTTC | V G N N D H I V A P P G Y H A F Y C H G E C P S H I | antagonist because it |
| | | AGTGACCTGGOGTGGAATGACTGGATTGTGGCTCCC | A G T S G S S L S P H S T L V M H Y R M R G H S P F | competes receptor binding |
| | | CCGGGGTATCACGCCTTTTACTGCCACGGAGAATGC | A N L K S C C V P T K I R P M S M L Y Y D D G Q N I | but not signaling. Acts |
| | | CCTTCTCATATAGCAGGCACGTCCGGGTCCTCACTGT | I K K D T Q N M I V E E C G C S | as neither BMP-2 nor |
| | | CCTTCCACTCAACGTTGGTCAACCACTACCGCATGCG | | activin-(IA. |
| | | GGGCCATAGCCCCTTTGCCAACCTCAAATCGTGCTGT | | |
| | | GTCCCGACCAAGCTGACACCCATGTCCATOTTGTACT | | |
| | | ATGATGATGGTCAAAACATCATCAAAAAGGACATTC | | |
| | | AGAACATGATCGTGGAGGAGTGTGGGTGCTCA | | |
| 1b2b3a4a5b6a | A32-002 | ATGCAAGCCAAACACAAACAGCGGAAACGCCTTAA | M Q A K H K Q R K P L K S S C E R H P L Y V D F S D | Activity in stem cell |
| | | GTCCAGCTGTAAGAGACACCCTTTGTACGTGGACTTC | V G M N D W I V A P P G Y H A P Y C H G E C P S B I | differentiation assays |
| | | AGTGACGTGGGCTGGAATGACTGGATTGTGGCTCCC | A G T S G S S L S I N S T I V N H Y R M R G H S P F | unlike BMP-2 |
| | | CGGGGGTATCACGCCTTTTACTGCCACGGAGAATGC | A N L E S C C V P T E L S A I S M L Y Y D D G Q N I I | |
| | | CCTTCTCATATAGGAGGCACGTCCGGGTCCTCACTGT | K K D I Q N M I V E E C G C S | |
| | | ccTTAGACTCAACGTTGGTCAACCACTACCGCATGCG | | |
| | | GGGCCATAGCCCCTTTGCCAACCTCAAATCGTGCTGT | | |
| | | GTCCCGACAGAGCTCAGTGCTATCTCGATGTTGTACT | | |
| | | ATGATGATOGTCAPAACATCATCAAAAAGGACATTC | | |
| | | AGAACATGATCGTGOAGGAGTGTGOGTGCTCA | | |
| 1b2a3a4a5b6a | AB2-003 | ATGCAAGCCAAACACAAACAGCGGAAACGCCTTAA | M Q A K H K Q R K R L K S S C K R H P L Y V D F S D | |
| | | GTCCAGCTGTAAGAGACACCCTTTGTAGGTGGACTTC | V G W N D W I T A P S G Y H A N Y C E G E C P S H I | |
| | | AGTGACGTGGGGTGGAATGACTGGATCATTGCTCCC | A G T S G S S L S F H S T L V N H Y R M R G H S P F | |
| | | TCTGGCTATCATGCCAACTACTGCGAGGGACAATGC | A N L K S C C V P T E L S A I S M I Y Y D D G Q N I I | |
| | | CCTTCTCATATAGCAGGCACGTCCGGGTCCTCACTGT | K K D I Q N M I V E E C G C S | |
| | | CCTTCCACTCAACGTTGGTCAACCACTACCGCATGCG | | |
| | | GGGCCATAGCCCCTTTGCCAACCTCAAATCGTGCTGT | | |
| | | GTCCCGACAGAACTCAGTGCTATCTCGATGTTGTACT | | |
| | | ATGATGATGGTCAAAACATCATCAAAAAGGACATTC | | |
| | | AGAACATGATCGTGGAGGAGTGTGGGTGCTCA | | |
| 1h2a3b4b5a6a | AB2-004 | ATGCAAGCCAAACACAAACAGCGGAAGCGTCTTAAG | M Q A K H K Q R K R L E S S C K R H P L Y V D F S D | ‘Super” BMP-2 |
| | | TCCAGCTGCAAAAGGCACCCTTTGTATGTGGACTTCA | V G W N D W I V A P S G Y H A N Y C D G E G P F P L | activity, unable to he |
| | | GTGATGTGGGGTGGAATGACTGGATCATTGCTCCCT | A D H I N S T N H A T V Q T I V M S V N S K I P K A | inhibited by Noggin |
| | | CTGGCTATCATGCCAACTACTGCGACGGAGAATGCC | C C V P T K L R P M S M L Y Y D D G Q N I I K K D I | |
| | | CTTTTCCTCTGGCTGATCATCTGAACTCCACTAATCA | Q N M I V E E C G C S | |
| | | TGCCATTGTTCAGACGTTGGTCAACTCTGTTAACTCT | | |
| | | AAGATTCCTAAGGCATGCTGTGTCCCGACCAAGCTG | | |
| | | AGACCCATGTCCATGTTGTACTATGATGATGGTCAA | | |
| | | AACATCATCAAAAAGGACATTCAGAACATGATCGTG | | |
| | | GAGGAGTOTGOGTGCTCA | | |
| 1b2b3b4b5b6b | AB2-005 | ATGCAAGCCAAACACAAACAGCGGAAACGCCTTAA | MQAKHKORKRLESSCERHPLYVDFSD | AB2-005 (BMP-2mq) |
| (BMP-2ₘₐ) | | GTCCAGCTGTAAGAGACACCCTTTGTACGTGGACTTC | VGVINDWIVAPPGYHAPYCHGECPFPL | contains one amino acid |
| | | AGTGACGTGGGGTGGAATGACTGGATTGTGGCTCCC | A D H L N S T N H A I V Q T L V N S V N S K I P K A | (Met) added at the N- |
| | | CCGGGGTATCACGCCTTTTACTGCCACGGAGAATGC | C C V P T E L S A I S M L Y L D E N E K V V L K N Y | terminus of mature BMP-2 |
| | | CCTTTTCCTCTGGCTGATCATCTGAACTCCACTAATC | QDMVVEGCGCR | in nature. Met originates |
| | | ATGCCATTGTTCAGACGTTGGTCAACTCTGTTAACTC | | from the translation |
| | | TAAGATTCCTAAGGCATGCTGTGTCCCGACAGAACT | | initiation codon (ATG). |
| | | CAGTGCTATCTCGATGCTGTACCTTGACGAGAATGA | | Unless it is truncated |
| | | AAAGGTTGTATTAAAGAACTATCAGGACATGGTTGT | | the N-terminus of AB2-00S. |
| | | GGAGGGTTGTGGGTGTCGC | | during the folding |
| 1b2a3a4a5b6b | AB2-006 | ATGCAAGCCAAACACAAACAGCGGAAACGCCTTAA | MOAKHKGRKRIKSSCKRHPLYVDFSD | Activity in stem cell |
| | | GTCCAGCTGTAAGAGACACCCTTTGTACGTGGACTTC | VGWNDWITAPSGYHANYCEGECPSHI | differentiation assays |
| | | AGTGACGTGGGGTGGAATGACTGGATCATTGCTCCC | AGTSGSSLSPHSTLVMHYRMAGHSPF | unlike BMP-2. |
| | | TCTGGCTATCATGCCAACTACTGCGAGGGAGAATGC | ANLKSCCVPTELSAISMLYLDENEKV | |
| | | CCTTCTCATATAGCAGGCACGTCCGGGTCCTCACTGT | VIKNYQDMVVEGCGCR | |
| | | CCTTCCACTCAACGTTGGTCAACCACTACCGCATGCG | | |
| | | GGGCCATAGCCCCTTTGCCAACCTCAAATCGTGCTGT | | |
| | | GTCCCGACAGAACTCAGTGCTATCTCGATGTTGTACC | | |
| | | TTGACGAGAATGAAAAGGTTGTATTAAAGAACTATC | | |
| | | AGGACATGGTTGTGGAGGGTTGTGGGTGTCGC | | |
| 1b2a2a4a5a6b | AB2-007 | ATGCAAGCCAAACACAAACAGCGGAAACGCCTTAA | MGAKHKQRKPLKSSCKRHPLYVDFSD | Activity in stem cell |
| | | GTCCAGCTGTAAGAGACACCCTTTGTACGTGGACTTC | VGWNDWITAPSGYHANYCEGSCPSHI | differentiation assays |
| | | AGTGACGTGGGGTGGAATGACTGGATCATTGCTCCC | AGTSGSSLSFHSTLVNHYRMRGHSPF | unlike BMP-2. |
| | | TCTGGCTATCATGCCAACTACTGCGAGGGAGAATGC | ANLKSCCVPTKLRPMSMLYLDENEKV | |
| | | CCTTCTCATATAGCAGGCACGTCCGGGTCCTCACTGT | VIKNYQDMVVEGCGCR | |
| | | CCTTCCACTCAACGTTGGTCAACCACTACCGCATGCG | | |
| | | GGGCCATAGCCCCTTTGCCAACCTCARATCGTCCTGT | | |
| | | GTCCCGACCAAGCTGAGACCCAMTCCATGTTGTAC | | |
| | | CTTGACGAGAATGAAAAGGTTGTATTAAAGAACTAT | | |
| | | CAGGACATGGTTGTOCAGGGTTGTGGGTGTCGC | | |
| 1b2a3a4a5a6a | AB2-008 | ATGCAAGCCAAACACAAACAGCGGAAACGCCTTAA | MQAKHKQRKRIKSSCKRHPLYVDFSD | Functions like activin- |
| | | GTCCAGCTGTAAGAGACACCCTTTGTACGTGGACTTC | VGWNDWITAPSGYHANYCEGECPSHI | AA in cell signaling and |
| | | AGTGACGTGGGGTGGAATGACTGGATCATTGCTCCC | AGTSGSSISPHSTIVNHYRMRGHSPF | in vivo experiments, ~4- |
| | | TCTGGCTATCATGCCAACTACTGCGAGGGAGAATGC | ANLKSCCVPTKIRPMSMLYYDDGQNI | fold lower potency; |
| | | CCTTCCCATATAGCAGGCACGTCCGGGTCCTCACTGT | IKKDIgNMIVESCGCS | replaces TOP-beta 1 in |
| | | CCTTCCATTCAACGTTGGTCAACCACTACCGCATGCG | | chemically-defined stem |
| | | GGGCCATAGCCCCTTTGCCAACCTCAAATCGTGCTGT | | cell media containing |
| | | GTCCCGACCAAGCTGAGACCCATGTCCATGTTGTACT | | FGF2. |
| | | ATGATGATGGTCAAAACATCATCAAAAAGGACATTC | | |
| | | AGAACATGATCGTGGAGGAGTGTGGGTGCTCA | | |
| 1o2a3a4aSa6a. | P22-009 | ATGCAAGCCAAACACAAACAGCGGAAACGCCTTAA | MQAKHKQRKRIKSSCKRHPLYVDFSD | Functions like activin- |
| L66V/V671 | | GTCCAGCTGTAAGAGACACCCTTTGTACGTGGACTTC | VGWNDWITAPSGYHANYCEGSCPSHI | AA in cell signaling |
| | | AGTGACGTGGGGTGGAATGACTGGATCATTGCTCCC | AGTSGSSISPHSTVINHYRMRGRSPFA | and in vivo experiments, |
| | | TCTGGCTATCATGCCAACTACTGCGAGGGAGAATGC | NIKSCCVPTKIRPMSMLYYDDGQNII | -10-fold lower potency |
| | | CCTTCCCATATADCAGGCACGTCCGGGTCCTCACTGT | KKDIQNMIVBECGCS | in activin-IIA signaling |
| | | CCTTCCATTCAAGGGTGATCAACCACTACCGCATGCG | | activity. |
| | | GGGCCATAGCCCCTTTGCCAACCTCAAATCGTGCTGT | | |
| | | GTCCCGACCAAGCTGAGACCCATGTCCATGTTGTACT | | |
| | | ATGATGATGGTCAAAACATCATCAPAAAGGACATTC | | |
| | | AGAACATGATCGTGGAGGAGTGTGGGTGCTCA | | |
| lb(la II) | AB2-010 | ATGCAAGCCAAACACAAACAGCGGAAACGCCTTAA | MQAKHKQKKALKSSCKKGPFVSFKDI | Functions like activin- |
| 2a3a4aSa6a | | GTCCAGCTGTAAGAAACAGTTCTTTGTCAGTTTCAAGG | WNDWITAPSGYHANYCEGECPSHIA | PA in cell signaling and |
| | | GACATCGGGTGGAATGACTGGATCATTGCTCCCTCT | GTSGSSISFHSTIVNHYRMAGHSPFA | in vivo experiments, ~20- |
| | | GGCTATCATGCCAACTACTGCGAGGGAGAATGCCCT | NLKSCCVPTKLRPMSMIYYDDGQNII | fold lower potency in |
| | | TCCCATATAGCAGGCACGTCCGGGTCCTCACTGTCCT | K K D I Q N M I V E E C G C S | activin-PA sionaling |
| | | TCCATTCAACGTTGGTCAACCACTACCGCATGCGGG | | |
| | | GCCATAGCCCCTTTGCCAACCTCAAATCGTGCTGTGT | | |
| | | CCCGACCAAGCTGAGACCCATGTCCATGTTGTACTAT | | |
| | | CATGATGGTCAAAACATCATCAAAAACGACATTCAG | | |
| | | AACATGATCGTGGAGGAGTGTGGGTGCTCA | | |
| 1b2b2b4b5a6a | AB2-011 | ATGCAAGCCAAACACCAACAGCGGAAACGCCTTAAG | MQAKEQQAKRLKSSCKKHPLYVDFSD | ‘Super” BMP-2 activity, |
| | | TCCAGCTGTAAGAGACACCCTTTGTACGTGGACTTCA | VGWNDWIVAPPGYHAPYCHGEGPFPL | unable to be inhibited by |
| | | GTGACGTGGGGTGGAATGACTGGATTGTGGCTCCCC | ADHLNSTNHAIVQTLVNSVNSKIPKA | Noggin |
| | | CGGGGTATCACGCCTTTTACTGCCACGGAGAATGCC | CCVPTKLAPSMLYYDDGQNIIKKDIQ | |
| | | CTTTTCCTCTGGCTGATCATCTGAACTCCACTAATCA | N M I V E E C G C S | |
| | | TGCCATTGTTCAGACGTTGGTCAACTCTGTTAACTCT | | |
| | | AAGATTCCTAAGGCATGCTGTGTCCCGACCAAGCTG | | |
| | | AGACCCTCCATGTTGTACTATGATGATGGTCAAAAC | | |
| | | ATCATCAAAAAGGACATTCAGAACATGATCGTGGAG | | |
| | | GAGTGTGGGTGCTCA | | |
| 1b2b2b4b5b6a | AB2-012 | ATGCAAGCCAAACACAAACAGCGGAAACGCCTTAA | MQAKHKQRKKLKSSCKRHPLYVDFSD | ‘Super” BMP-2 activity, |
| | | GTCCAGCTGTAAGAGACACCCTTTGTACGTGGACTTC | VGLINDWIVAPPGYHAPYCHGEGPFP1 | partially inhibited by |
| | | AGTGACGTGGGGTGGAATGACTGGATTGTGGCTCCC | ADHLNSTNHAIVQTIVNSVNSKIPKA | Noggin |
| | | CCGGGGTATCACGCCTTTTACTGCCACGGAGAATGC | CCVPTELSAISMLYYDDGRNIIKKDIQ | |
| | | CCTTTTCCTCTGGCTGATCATCTGAACTCCACTAATC | NMIVEECGCS | |
| | | ATGCCATTGTTCAGACGTTGOTCAACTCTGTTAACTC | | |
| | | TAAGATTCCTAAGGCATGCTGTGTCCCGACAGAACT | | |
| | | CAGTGCTATCTCGATGTTGTACTATGATGATGGTCGA | | |
| | | AACATCATCAAAAAGGACATTCAGAACATGATCGTG | | |
| | | GAGGAGTGTGGGTGCTCA | | |
| 1b2b3b4b5a6b | AB2-013 | ATGCAAGCCAAACACAAACAGCGGAAACGCCTTAA | MQAKHKQRKALKSSCKRHPLYVDFSD | Activity comparable to |
| | | GTCCAGCTGTAAGAGACACCCTTTGTACGTGGACTTC | VGWNDWIVAPPGYHAFYCHGECPEPL | BMP-2, inhibited by |
| | | AGTGACGTGGGGTGGAATGACTGGATTGTGGCTCCC | A D H L N S T N H A I V Q T L V N S V N S K I P K A | Noggin |
| | | CCGGGGTATCACGCCTTTTACTGCCACGGAGAATGC | CCVPTKLRPMSMLYYDENEKVVLKN | |
| | | CCTTTTCCTCTGGCTGATCATCTGAACTCCACTAATC | YQDMVVECCGCR | |
| | | ATGCCATTGTTCAGACGTTGGTCAACTCTGTTAACTC | | |
| | | TAAGATTCCTAAGGCATGCTGTGTCCCGACCAAGCT | | |
| | | GAGACCCATGTCCATGTTGTACTATGATOAGAATGA | | |
| | | AAAGGTTGTATTAAAGAACTATCAGGACATGGTTGT | | |
| | | GGAGGGTTGTGGGTGTCGC | | |
| 1b2a3b4b5a6b | AB2-014 | ATGCAAGCCAAACACAAACAGCGGAAACGCCTTAA | MQAKHKQKKALKSSCKREPLYVDFSD | Activity comparable to |
| | | GTCCAGCTGTAAGAGACACCCTTTCTACGTGGACTTC | VGWNDWITAPSCYHANYCDGECPFPL | BMP-2, partially blocked |
| | | AGTGACGTGGGGTGGAATGACTGGATCATTGCTCCC | ADHLNSTNHAIVQTIVNSVNSKTPKA | by Noggin |
| | | TCTGGCTATCATGCCAACTACTGCGACGGAGAATGC | CCVPTKIRPMSMIYIDENEKVVLKNY | |
| | | CCTTTTCCTCTGGCTGATCATCTGAACTCCACTAATC | Q D M V V E G C G C R | |
| | | ATGCCATTGTTCAGACGTTGGTCAACTCTGTTAACTC | | |
| | | TAAGATTCCTAAGGCATGCTGTOTCCCGACCAAGCT | | |
| | | GAGACCCATGTCCATGTTGTACCTTGACGAGAATGA | | |
| | | AAAGGTTGTATTAAAGAACTATCAGGACATGGTTGT | | |
| | | GGAGGGTTGTGGGTGTCGC | | |
| 1b2a3b4b5b6a | AB2-015 | ATGCAAGCCAAACACAAACAGCGCAAACGCCTTAA | M Q A K H K Q R K R L K S S C K R H P L Y V D F S D | ‘Super” BMP-2 activity, |
| | | GTCCAGCTGTAAGAGACACCCTTTGTACGTGGACTTC | VGWEDWITAPSGYHANYCDGECPFPL | unable to be inhibited by |
| | | AGTGACGTGGGGTGGAATGACTGGATCATTGCTCCC | ADNINSTNHAIVQTIVNSVNSKIPEA | Noggin |
| | | TCTGGCTATCATGCCAACTACTGCGACGGAGAATGC | CCVPTELSAISMLYYDDGQNIIKKDIQ | |
| | | CCTTTTCCTCTGGCTGATCATCTGAACTCCACTAATC | N M I V E E C G C S | |
| | | ATGCCATTGTTCAGACGTTGOTCAACTCTOTTAACTC | | |
| | | TAAGATTCCTAAGGCATGCTGTGTCCCGACAGAACT | | |
| | | CAGTGCTATCTCGATGTTGTACTATGATGATGGTCAA | | |
| | | AACATCATCAAAAAGGACATTCAGAACATGATCGTG | | |
| | | GAGGAGTGTGGGTGCTCA | | |
| lb,2a3b4bSb6b | AB2-016 | ATGCAAGCCAAACACAAACAGCGGAAGCGTCTTAAG | MQAKHKQRKRLKSSCKNHPLYVDFSD | Activity comparable to |
| | | TCCAGCTGCAAAAGGCACCCTTTGTATGTGGACTTCA | VGWNDWITAPSGYHANYCEGECPFP1 | BMP-2, inhibited by |
| | | GTGATGTGGGGTGGAATGACTGGATCATTGCTCCCT | ADHINSTNHAIVQTIVNIVNSKIPKA | Noggin |
| | | CTGGCTATCATGCCAACTACTGCGAGGGAGAATGCC | CCVPTELSAISMLYLDENEKVVLKNY | |
| | | CTTTTCCTCTGGCTGATCATCTGAACTCCACTAATCA | Q D M V V E G C G C R | |
| | | CGCCATTGTTCAGACGTTGGTCAACTCTGTTAACTCT | | |
| | | AAGATTCCTAAGGCATGCTGTGTCCCGACAGAACTC | | |
| | | AGTGCTATCTCGATGCTGTACCTTGACGAGAATGAA | | |
| | | AAGGTTGTATTAAAGAACTATCAGGACATGOTTGTG | | |
| | | GAGGGTTGCGGGTGTCGT | | |
| 1b2b3b4a5a6a | AB2-017 | ATGCAAGCCAAACACAAACAGCGGAAACGCCTTAA | MQAKHKQRKRIKSSCKRHPLYVDFSD | |
| | | GTCCAGCTGTAAGAGACACCCTTTGTACGTGGACTTC | VGWNDWIVAPPGYHAPYCHGECPFPL | |
| | | AGTGACGTGGCGTGCAATCACTCCATTGTGGCTCCC | ADHLNSTNHAIVQTLVNHYRMRGHSP | |
| | | CCGGGGTATCACGCCTTTTACTGCCACGGAGAATGC | FANLKSCCVPTKLRPMSMLYYDDGQN | |
| | | CCTTTTCCTCTGGCTGATCATCTGAACTCCACTAATC | TIKKDIQNMIVEECGCS | |
| | | ATGCCATTGTTCAGACGTTGGTCAACCACTACCGCAT | | |
| | | GCGGGGCCATAGCCCCTTTGCCAACCTCAAATCGTG | | |
| | | CTGTGTCCCGACCAAGCTGAGACCCATOTCCATGTTG | | |
| | | TACTATGATGATGGTCAAAACATCATCAAAAAGGAC | | |
| | | ATTCAGAACATGATCGTGGAGGAGTGTGGGTGCTCA | | |
| 1b2b3b4a5b6b | AB2-018 | ATGCAAGCCAAACACAAACAGCGGAAACGCCTTAA | MOAKHKQRKRIKSSCRRHPLYVDPSD | |
| | | GTCCAGCTGTAAGAGACACCCTTTGTACGTGGACTTC | VGMNDWIVAPPGYHAFICHGECPFPL | |
| | | AGTGACGTGGGGTGGAATGACTGGATTGTGGCTCCC | ADHLMSTNHAIVQTLVNHYRMRGHSP | |
| | | CCGGGGTATCACGCCTTTTACTGCCACGGAGAATGC | FANLKSCCVPTELSAISTLYLDENEKV | |
| | | CCTTTTCCTCTGGCTGATCATCTGAACTCCACTAATC | VIKNYQDMVVEGCGCR | |
| | | ATGCCATTGTTCAGACGTTGGTCAACCACTACCGCAT | | |
| | | GCGGGGCCATAGCCCCTTTGCCAACCTCAAATCGTG | | |
| | | CTGTGTCCCGACAGAACTCAGTGCTATCTCGATACTG | | |
| | | TACCTTGACGAGAATGAAAAGGTTGTATTAAAGAAC | | |
| | | TATCAGGACATGGTTGTGGAGGGTTGTGGGTGTCGC | | |
| 1b2b3b4a5a6b | AB2-019 | ATGCAAGCCAAACACAAACAGCGGAAACGCCTTAA | M Q A K H K Q R K R L K S S C K R H P L Y V D F S D | |
| | | GTCCAGCTGTAAGAGACACCCTTTGTACGTGGACTTC | VGWNDWIVAPPGYHAFYCHGEGPFPL | |
| | | AGTGACGTGGGGTGGAATGACTGGATTGTGGCTCCC | ADHLNSTNEAIVQTLVNHYRMRGHSP | |
| | | CCGGGGTATCACGCCTTTTACTGCCACGGAGAATGC | FANLKSCCVPTKLRPMSMLYYDENEK | |
| | | CCTTTTCCTCTGGCTGATCATCTGAACTCCACTAATC | V V L K N Y Q D M V V E G C G C R | |
| | | ATGCCATTGTTCAGACGTTGGTCAACCACTACCGCAT | | |
| | | GCGGGGCCATAGCCCCTTTGCCAACCTCAAATCGTG | | |
| | | CTGTGTCCCGACCAAGCTGAGACCCATGTCCATGTTG | | |
| | | TACTATGATGAGAATGAAAAGGTTGTATTAAAGAAC | | |
| | | TATCAGGACATGGTTGTGGAGGGTTGCGGGTGTCGT | | |
| 1b2b3b4a5b6a | AB2-020 | ATGCAAGCCAAACACAAACAGCGGAAACGCCTTAA | M Q A K H K Q R K R L K S S C K R H P L Y V D F S D | |
| | | GTCCAGCTGTAAGAGACACCCTTTGTACGTGGACTTC | VGWNDWIVAPPGYHAFYCHGEGPFPL | |
| | | AGTGACGTGGGGTGGAATGACTGGATTGTGGCTCCC | A D H L N S T N H A I V Q T L V N H Y R M R G H S P | |
| | | CCGGGGTATCACGCCTTTTACTGCCACGGAGAATGC | F A N C K S C C V P T E L S A I S M L Y Y D D G Q N I | |
| | | CCTTTTCCTCTGGCTGATCATCTGAACTCCACTAATC | I K K D I Q N M I V E E C G C S | |
| | | ATGCCATTGTTCAGACGTTGGTCAACCACTACCGCAT | | |
| | | GCGGGGCCATAGCCCCTTTGCCAACCTCAAATCGTG | | |
| | | CTGTGTCCCGACAGAACTCAGTGCTATCTCGATGTTG | | |
| | | TACTATGATGATGGTCAAAACATCATCAAAAAGGAC | | |
| | | ATTCAGAACATGATCGTGGAGGAGTGTGGGTGCTCA | | |
| 1b2b3a4a5a6b | AB2-021 | ATGCAAGCCAAACACAAACAGCGGAAACGCCTTAA | MQAKHKQRKALKSSCKRHPLYVDFSD | |
| | | GTCCAGCTGTAAGAGACACCCTTTGTATGTGGACTTC | VGWNDWIVAPPGYHAPYCHGECPSRI | |
| | | AGTGACGTGGGGTGGAATGACTGGATTGTGGCTCCC | AGTSGSSLSEHSTLVNHYRMRGHSPF | |
| | | CCGGGGTATCACGCCTTTTACTGCCACGGAGAATGC | A N L K S C C V P T K L R P M S M L Y L D E N E K V | |
| | | CCTTCTCATATAGCAGGCACGTCCGGGTCCTCACTGT | V L K N Y Q D M V V E G C G C R | |
| | | CCTTCCACTCAACGTTGGTCAACCACTACCGCATGCG | | |
| | | GGGCCATAGCCCCTTTGCCAACCTCAAATCGTGCTGT | | |
| | | GTCCCGACCAAGCTGAGACCCATGTCCATGTTGTAC | | |
| | | CTTGACGAGAATGAAAAGGTTGTATTAAAGAACTAT | | |
| | | CAGGACATGGTTGTGGAGGGTTGTGGGTGTCGC | | |
| 1b2b3a4a5b6b | AB2-022 | ATGCAAGCCAAACACAAACAGCGGAAACGCCTTAA | MQAKHKQRKRLKSSCKREPLYVDFSD | |
| | | GTCCAGCTGTAAGAGACACCCTTTGTACGTGGACTTC | VGWEDWIVAPPGYHAFYCHGECPSHI | |
| | | AGTGACGTGGGGTGGAATGACTGGATTGTGGCTCCC | AGTSGSSLSFESTLVNHYRMRGHSPF | |
| | | CCGGGGTATCACGCCTTTTACTGCCACGGAGAATGC | A N L K S C C V P T E L S A I S M L Y L D E N E K V | |
| | | CCTTCTCATATAGCAGGCACGTCCGGGTCCTCACTGT | V L K N Y Q D M V V E G C G C R | |
| | | CCTTCCACTCAACGTTGGTCAACCACTACCGCATGCG | | |
| | | GGGCCATAGCCCCTTTGCCAACCTCAAATCGTGCTGT | | |
| | | GTCCCGACAGAACTCAGTGCTATCTCGATGTTGTACT | | |
| | | ATGATGAGAATGAAAAGGTTGTATTAAAGAACTATC | | |
| | | AGGACATGGTTGTGGAGGGTTGC | | |
| 1b2b3a4b5b6b | AB2-023 | ATGCAAGCCAAACACAAACAGCGGAAACGCCTTAA | MQAKHKQRKELKSSCKRHPLYVDFSD | |
| | | GTCCAGCTGTAAGAGACACCCTTTGTACOTGGACTTC | VGWHDWIVAPPGYHAFYCHGECPSHI | |
| | | AGTGACGTGGGGTGGAATGACTGGATTGTGGCTCCC | A G T S G S S L S F H S T L V N S V N S K I P K A C C | |
| | | CCGGGGTATCACGCCTTTTACTGCCACGGAGAATGC | VPTELSAISMLYLDENEKVVLKNYOD | |
| | | CCTTCTCATATAGCAGGCACGTCCGGGTCCTCACTGT | MVVEGCGCK | |
| | | CCTTCCACTCAACGTTGGTCAACTCTGTTAACTCTAA | | |
| | | GATTCCTAAGGCATGCTGTGTCCCCACAGAACTCAG | | |
| | | TGCTATCTCGATGCTGTACCTTGACGAGAATGAAAA | | |
| | | GGTTGTATTAAAGAACTATCAGGACATGGTTGTGGA | | |
| | | GGGTTGCOGGTGTCGT | | |
| 1b2b3a4b5b6a | A32-024 | ATGCAAGCCAAACACAAACAGCGGAAACGCCTTAA | MQAKHKQRKALKSSCKRHPLYVDFSD | |
| | | GTCCAGCTGTAAGAGACACCCTTTGTACGTGGACTTC | V G W N D W I V A P P G Y H A F Y C H G E C P S H I | |
| | | AGTGACGTGGGGTGGAATGACTGGATTGTGGCTCCC | AGTSCSSLSPHSTLVNSVNSKIPKACC | |
| | | CCGGGGTATCACGCCTTTTACTGCCACGGAGAATGC | VPTELSAISMLYYDDCONIIKKDIONM | |
| | | CCTTCTCATATAGCAGGCACGTCCGGGTCCTCACTGT | I V E E C G C S | |
| | | CCTTCCACTCAACGTTGGTCAACTCTGTTAACTCTAA | | |
| | | GATTCCTAACCCATGCTGTGTCCCGACAGAACTCAG | | |
| | | TGCTATCTCGATGTTGTACTATGATGATGGTCAAAAC | | |
| | | ATCATCAAAAAGGACATTCAGAACATGATCGTGGAG | | |
| | | GAGTGTGGGTGCTCA | | |
| 1b2b3a4b5a6a | A22-025 | ATGCAAGCCAAACACAAACAGCGGAAACGCCTTAA | MQAKHKORKALKSSCKRHPLYVDFSD | |
| | | GTCCAGCTGTAAGAGACACCCTTTGTACGTGGACTTC | VGWNDWIVAPPGYHAFYCHGECPSHI | |
| | | AGTGACGTGGGGTGGAATGACTGGATTGTGGCTCCC | A G T S G S S L S F H S T L V N S V N S K I P K A C C | |
| | | CCGGGGTATCACGCCTTTTACTGCCACGCAGAATCC | V P T K L R P M S M L Y Y D D C Q N I I K K D I Q N | |
| | | CCTTCTCATATAGCAGGCACGTCCGGGTCCTCACTGT | M I V E E C G C S | |
| | | CCTTCCACTCAACGTTGGTCAACTCTGTTAACTCTAA | | |
| | | GATTCCTAAGGCATGCTGTGTCCCGACCAAGCTGAG | | |
| | | ACCCATGTCCATOTTGTACTATGATGATCGTCAAAAC | | |
| | | ATCATCAAAAAGGACATTCAGAACATGATCGTGGAG | | |
| | | GAGTGTGGGTGCTCA | | |
| 1b2b3a4b5a6b | A22-026 | ATGCAAGCCAAACACAAACAGCGGAAACGCCTTAA | MCAKHKQRKALKSSCKRHPLYVDFSD | |
| | | GTCCAGCTGTAAGAGACACCCTTTGTACGTGGACTTC | VOWNDWIVAPPGYHARYCHGECPSHI | |
| | | AGTGACGTGGGGTGGAATGACTGGATTGTGGCTCCC | AGTSGSSLSFHSTLVNSVNSKIPKACC | |
| | | CCGGGGTATCACGCCTTTTACTGCCACGGAGAATGC | VPTKLRPRSMLYYDENEKVV5KNYQ | |
| | | CCTTCTCATATAGCAGGCACGTCCGGGTCCTCACTGT | DMVVEGCGCR | |
| | | CCTTCCACTCAACGTTGGTCAACTCTGTTAACTCTAA | | |
| | | GATTCCTAAGGCATGCTGTGTCCCGACCAAGCTGAG | | |
| | | ACCCATGTCCATGTTGTACTATGATGAGAATGAAAA | | |
| | | GGTTGTATTAAAGAACTATCAGGACATGGTTGTGGA | | |
| | | GGGTTGCGGGTGTCGT | | |
| 1b2a3a4b5b6b | A22-027 | ATGCAAGCCAAACACAAACAGCGGAAACGCCTTAA | MOAKHKQRKALKSSCKRHPLYVDFSD | |
| | | GTCCAGCTGTAAGAGACACCCTTTGTACGTGGACTTC | VGWNDWIIAPSGYHANYCEGECPSHI | |
| | | AGTGACGTGGGGTGGAATGACTGGATCATTGCTCCC | AGTSGSSLSPHSTLVNSVHSKIPKACC | |
| | | TCTGGCTATCATGCCAACTACTGCGAGGGAGAATGC | VPTELSAISMLYLDENEKVVLKNYQD | |
| | | CCTTCTCATATAGCAGGCACGTCCGGGTCCTCACTGT | M V V E G C G C R | |
| | | CCTICCACTCAACGTTGGTCAACTCTGTTAACTCTAA | | |
| | | GATTCCTAAGGCATGCTGTGTCCCGACAGAACTCAG | | |
| | | TGCTATCTCGATGTTGTACCTTGACGAGAATGAAAA | | |
| | | GGTTGTATTAAAGAACTATCAGGACATGGTTGTGGA | | |
| | | GGGTTGTGGGTGTCGC | | |
| 1b2a3a4b5b6a | AB2-020 | ATGCAAGCCAAACACAAACAGCGGAAACGCCTTAA | MQAKHKQEKRIKSSCKRHPLYVDFSD | |
| | | GTCCAGCTGTAAGAGACACCCTTTGTACGTGGACTTC | VGWNDWITAPSGYHANYCEGECPSHI | |
| | | AGTGACGTGGGGTGGAATGACTGGATCATTGCTCCC | AGTSGSSLSFNSTLVNSVNSKIPKACC | |
| | | TCTGGCTATCATGCCAACTACTGCGAGGGAGAATGC | V P T E L N A I S M L Y Y D D G Q N I I K K D I Q N | |
| | | CCTTCTCATATAGCAGGCACGTCCGGGTCCTCACTGT | M I V E E C G C S | |
| | | CCTTCCACTCAACGTTGGTCAACTCTGTTAACTCTAA | | |
| | | GATTCCTAAGGCATGCTGTGTCCCGACAGAACTCAA | | |
| | | TGCTATCTCOATGTTGTACTATGATGATGOTCAAAAC | | |
| | | ATCATTAAAAAGGACATTCAGAACATGATCGTGGAG | | |
| | | GAGTGTGGGTGCTCA | | |
| 1b2a3a4b5a6a | AB2-030 | ATGCAAGCCAAACACAAACAGCGGAAACGCCTTAA | MQAKHKQEKRIKSSCKRHPLYVDFSD | |
| | | GTCCAGCTGTAAGAGACACCCTTTGTACGTGGACTTC | VGWNDWITAPSGYHANYCEGECPSHI | |
| | | AGTGACGTGGGGTGGAATGACTGGATCATTGCTCCC | AGTSGSSISFHSTLVMSVNSKIPKACC | |
| | | TCTGGCTATCATGCCAACTACTGCGAGGGAGAATGC | VPTKIRPMSMLYYDDGQNIIKKDIQN | |
| | | CCTTCTCATATAGCAGGCACGTCCGGGTCCTCACTGT | M I V E E C G C S | |
| | | CCTTCCACTCAACGTTGGTCAACTCTGTTAACTCTAA | | |
| | | GATTCCTAAGGCATGCTGTGTCCCGACCAAGCTGAG | | |
| | | ACCCATGTCCATGTTGTACTATGATGATGGTCAAAAC | | |
| | | ATCATCAAAAAGGACATTCAGAACATGATCGTGGAG | | |
| | | GAGTGTGGGTGCTCA | | |
| 1b2a3b4a5a6a | AB2-031 | ATGCAAGCCAAACACAAACAGCGGAAACGCCTTAA | MQAKHKQRKRLKSSCKRNPLYVDFSD | |
| | | GTCCAGCTGTAAGAGACACCCTTTGTACGTGGACTTC | V G W N D W I I A P S G Y H A N Y C E G E C P F P L | |
| | | AGTGACGTGGGGTGGAATGACTGGATCATTGCTCCC | ADHLNSTKHAIVQTLVNHYRMRGHSP | |
| | | TCTGGCTATCATGCCAACTACTGCGAGGGAGAATGC | FANLKSCCVPTKLRPMSMLYYDDGQN | |
| | | CCTTTTCCTCTGGCTGATCATCTGAACTCCACTAATC | I I K K D I Q N M I V E E C G C S | |
| | | ATGCCATTGTTCAGACGTTGGTCAACCACTACCGCAT | | |
| | | GCGGGGCCATAGCCCCTTTGCCAACCTCAAATCGTG | | |
| | | CTGTGTOCCGACCAAGCTGAGACCCATGTCCATGTTG | | |
| | | TACTATGATGATGOTCAAAACATCATCAAAPAGGAC | | |
| | | ATTCAGAACATGATCGTGGAGGAGTGTGGGTGCTCA | | |
| 1b2a3b4a5b6a | AB2-032 | ATGCAAGCCAAACACAAACAGCGGAAACGCCTTAA | MQAKHKQRKALKSSCKRHPLYVDPSD | |
| | | GTCCAGCTGTAAGAGACACCCTTTGTACGTGGACTTC | VGWNDWITAPSGYHANYCEGECPFPL | |
| | | AGTGACGTGGGGTGGAATGACTGGATCATTGCTCCC | A D H L N S T N H A I V Q T L V N H Y R M R G H S P | |
| | | TCTGGCTATCATGCCAACTACTGCGAGGGAGAATGC | FANLKSCCVPTELSAISMLYYDDGQN I | |
| | | CCTTTTCCTCTGGCTGATCATCTGAACTCCACTAATC | I K K D I 0 N M I V E E C G C S | |
| | | ATGCCATTGTTCAGACGTTGGTCAACCACTACCGCAT | | |
| | | GCGGGGCCATAGCCCCTTTOCCAACCTCAAATCGTG | | |
| | | CTGTGTCCCGACAGAACTCAGTGCTATCTCGATGCTG | | |
| | | TACCTTGACGATGGTCAAAACATCATCAAAAAGGAC | | |
| | | ATTCAGAACATGATCGTGGAGGAGTGTGGGTGCTCA | | |
| 1b2a3b4a5b6b | AB2-033 | ATGCAAGCCAAACACAAACAGCGGAAACGCCTTAA | MQAKHKQRKELKSSCKRHPLYVDPSD | |
| | | GTCCAGCTGTAAGAGACACCCTTTGTACGTGGACTTC | V G W N D W I I A P S G Y H A N Y C E G E C P F P L | |
| | | AGTGACGTGGGGTGGAATGACTGGATCATTGCTCCC | ADHLNSTNHAIVQTLVNHYRNIRGHSP | |
| | | TCTGGCTATCATGCCAACTACTGCGAGGGAGAATGC | FANLKSCCVPTELSAISMLYLDENEKV | |
| | | CCTTTTCCTCTGGCTGATCATCTGAACTCTACTAATC | V L K N Y Q D M V V E G C G C R | |
| | | ATGCCATTGTTCAGACGTTGGTCAACCACTACCGCAT | | |
| | | GCGGGGCCATAGCCCCTTTGCCAACCTCAAATCGTG | | |
| | | CTGTGTCCCGACAGAACTCAGTGCTATCTCGATGCTG | | |
| | | TACCTTGACGAGAATGAAAAGGTTGTATTAAAGAAC | | |
| | | TATCAGGACATGCTTGTGGAGGGTTGCGGGTGTCCT | | |
| 1b2a3b4a5a6b | AB2-034 | ATGCAAGCCAAACACAAACAGCGGAAGCGTCTTAAG | MQAKHKQEKELKSSCICREPLYVDFSD | |
| | | TCCAGCTGCAAAAGGCACCCTTTGTATGTGGACTTCA | VGWNDWITAPSGYHANYCDGECPFPL | |
| | | GTGATGTGGGGTGGAATGACTGGATCATTGCTCCCT | ADHLNSTNHAIVQTLVNHYRMEGHSP | |
| | | CTGGCTATCATGCCAACTACTGCGACGGAGAATGCC | FANLKSCCVPTKLRPMSNLYYDENEK | |
| | | CTTTTCCTCTGGCTGATCATCTGAACTCCACTAATCA | V V L K N Y Q D M V V E G C G C R | |
| | | TGCCATTGTTCAGACGTTGGTCAACCACTACCGCATG | | |
| | | CGGGGCCATAGCCCCTTTGCCAACCTCAAATCATGCT | | |
| | | GTGTCCCGACCAAGCTGAGACCCATGTCCATGTTGT | | |
| | | ACTATGATGAGAATGAAAAGGTTGTATTAAAGAACT | | |
| | | ATCAGGACATGGTTGTGGAGGGTTGCGGGTGTCGT | | |
| BMP-2ₘₐ | 1314P-2ₘₐ | ATGGCTCAAGCCAAACACAAACAGCGGAAACGCCTT | MAQAKHKQRKELKSSCKSHPLYVDFS | BMP-2m, contains two |
| | | AAGTCCAGCTGTAAGAGACACCCTTTGTACGTGGAC | DVGNNDWIVAPPGYHAFYCHGECPFP | additional amino acids |
| | | TTCAGTGACGTGGGGTGGAATGACTGGATTGTGGCT | L A D H L N S T N H A I V Q T L V N S V N S K I P K | (Met-Ala) at the |
| | | CCCCCGGGGTATCACGCCTTTTACTGCCACGGAGAA | ACCVPTELSAISFILYLDENEKVVLKN | N-terminal side of |
| | | TGCCCTTTTCCTCTGGCTGATCATCTGAACTCCACTA | Y Q D M V V E G C G C R | mature BMP-2 in nature |
| | | ATCATGCCATTGTTCAGACGTTGGTCAACTCTGTTAA | | (QAKH . . . ). Met is |
| | | CTCTAAGATTCCTAAGGCATGCTGTGTCCCGACAGA | | often truncated during |
| | | ACTCAGTGCTATCTCGATGCTGTACCTTGACGAGAAT | | the folding process, |
| | | GAAAAGGTTGTATTAAAGAACTATCAGGACATGGTT | | but Ala remains |
| | | GTGGAGGGTTGTGGGTGTCGC | | as the N-terminus |
| | | | | of the final product. |
| | | | | Either form is |
| | | | | regarded as |
| | | | | BMP-2m,. |
| 1b_BMP7 | NB2-BMP7 | ATGCAAGCCAAACACAAACAGCGGAAACGCCTTAA | M Q A K H K Q R K R L K S S C K R H P L Y V D F S D | |
| | | GTCCAGCTGTAAGAGACACCCTTTGTACGTGGACTTC | VGWNDWITAPEGYAAYYCEGECAPPL | |
| | | AGTGACGTGGGGTGGAATGACTGGATTATCGCGCCT | N S Y M N A T N H A I V Q T L V H F I N P E T V P K | |
| | | GAAGGCTACGCCGCCTACTACTGTGAGGGGGAGTGT | PCCAPTQLNAISVLYFDDSSNVILKKY | |
| | | GCCTTCCCTCTGAACTCCTACATGAACGCCACCAACC | R N M V V R A C G C H | |
| | | ACGCCATCGTGCAGACGCTGGTCCACTTCATCAACC | | |
| | | CGGAAACGGTGCCCAAGCCCTGCTGTGCGCCCACGC | | |
| | | AGCTCAATGCCATCTCCGTCCTCTACTTCGATGACAG | | |
| | | CTCCAACGTCATCCTGAAGAAATACAGAAACATGGT | | |
| | | GGTCCGGGCCTGTGGCTGCCAC | | |
| 1b_BMP9 | NB2-BMP9 | ATGCAAGCCAAACACAAACAGCGGAAACGCCTTAA | M Q A K H K Q R K R L K S S C K R H P L Y V D F S D | |
| | | GTCCAGCTGTAAGAGACACCCTTTGTACGTGGACTTC | VGWNDWITAPKEYEAYECKGGCFFPL | |
| | | AGTGACGTGGGGTGGAATGACTGGATTATTGCCCCA | A D D V T P T K H A I V Q T L V H L K F P T K V G K | |
| | | AAAGAGTACGAGGCATACGAGTGTAAGGGCGGCTGT | A C C V P T K L S P I S V L Y K D D M G V P T L K Y | |
| | | TTCTTTCCGCTGGCCGACGATGTCACCCCGACCAAGC | H Y E G M S V A E C G C R | |
| | | ACGCAATTGTCCAAACCTTAGTGCACCTGAAGTTCCC | | |
| | | AACGAAAGTGGGTAAGGCATGTTGTGTGCCAACCAA | | |
| | | GTTATCTCCAATTAGCGTGCTGTATAAGGATGATATG | | |
| | | GGCGTGCCGACGTTAAAGTATCATTACGAGGGCATG | | |
| | | AGCGTCGCAGAGTGTGGCTGCCGC | | |
| 1b_GDF7 | NB2-GDF7 | ATGCAAGCCAAACACAAACAGCGGAAACGCCTTAA | M Q A K H K Q R K R L K S S C K R H P L Y V D F S D | |
| | | GTCCAGCTGTAAGAGACACCCTTTGTACGTGGACTTC | V G W N D W I I A P L D Y E A Y H C E G L C D F P L | |
| | | AGTGACGTGGGGTGGAATGACTGGATTATCGCGCCG | RSHLEPTNHAIIQTLVNSMAPDAAPAS | |
| | | CTGGACTACGAGGCGTACCACTGCGAGGGCCTATGC | CCVPARLSPISILYYDAANNVVYKQY | |
| | | GATTTTCCTCTGCGTTCGCACCTCGAACCCACCAACC | E D M V V E A C G C R | |
| | | ATGCCATCATTCAGACGTTGGTCAACTCCATGGCACC | | |
| | | AGACGCGGCGCCGGCCTCCTGCTGTGTCCCGGCGCG | | |
| | | CCTCAGCCCCATCAGCATCTTGTACTATGATGCCGCC | | |
| | | AACAACGTTGTCTACAAGCAATACGAGGACATGGTG | | |
| | | GTGGAGGCCTGTGGGTGTCGC | | |
| 1b_GDF8 | NB2-GDF8 | ATGCAAGCCAAACACAAACAGCGGAAACGCCTTAA | MQAKHKQRKRLKSSCKRHPLYVDFSD | |
| | | GTCCAGCTGTAAGAGACACCCTTTGTACGTGGACTTCV | G W N D W I I A P K R Y K A N Y C S G E C E F V F | |
| | | AGTGACGTGGGOTGCAATGACTGGATTATTGCACCC | LQKYPHTHLVHQANPRGSAGPCCTPT | |
| | | AAAAGATATAAGGCCAATTACTGCTCTGGAGAGTGT | KMSPINMLYRNGKEQITYGRIPAMVV | |
| | | GAATTTGTATTTTTACAAAAATACCCTCACACTCATC | DRCCCS | |
| | | TTGTOCACCAACCAAACCCCAGAGGTTCAGCAGGCC | | |
| | | CCTGCTGTACTCCCACAAAGATGTCTCCAATCAATAT | | |
| | | GCTATATTTTAATGGCAAAGAACAAATAATATATGG | | |
| | | GAAAATTCCAGCCATCGTAGTAGATCGCTGTGCGTG | | |
| | | CTCA | | |
| BMP2/DMR6 | B2/B6 | BMP-2_{wt} and BMP-6 are added | BMP-2_{wt} sequence is reported above as AB2-005 BMP-6 | Has increased SMAD- |
| | | together during the refolding to | amino acid sequence. Two amino acids, MA, are | mediated signaling |
| | | generate the BMP2/BMP6 heterodimer. | present in BMP-2_{wt} in ocntrast to mature | activity as compared to |
| | | DMP-2_{wt} sequence herein is also | form of BMP-2 existent in nature. | either BMP-2 or BMP-6. |
| | | reported as AB2-000. BMP-6 DNA | MQQSRARSTQSQDVARVSSASDYNS5 | |
| | | sequence is: | ELKTACRKHELYVSFQDLGEQDWITA | |
| | | ATGCAACAGAGTCGTAATCGCTCTACCCAGTCCCAC | PEGYAANYCDCECSFPLNARMNATN | |
| | | GACGTCGCGCGGGTCTCCAGTGCTTCAGATTACAAC | HAIVQTLVHLMNPEYVPKPCCAPTKL | |
| | | AGCAGTGAATTGAAAACAGCCTGCACCAAGCATGA | NAISVLYFDDNSNVILKKYRNMVVRA | |
| | | GCTGTATGTGAGTTTCCAAGACCTGGGATCCCAGGA | C G C H | |
| | | CTGGATCATTGCACCCAAGGGCTATGCTGCCAATTA | | |
| | | CTGTGATGGAGAATGCTCCTTCCCACTCAACGCACA | | |
| | | CATGAATGCAACCAACCACGCGATTGTGCAGACCTT | | |
| | | GGTTCACCTTATGAACCCCGAGTATGTCCCCAAACC | | |
| | | GTGCTGTGCGCCAAcTAAGCTAAATGCCATCTCGGTT | | |
| | | CTTTACTTTCATGACAACTCCAATGTCATTCTGAAAA | | |
| | | AATACAGGAATATGGTTCTAAGAGCTTGTGGATGCC | | |
| | | AC | | |
| 1b2a3a4a5a6ab | AB2-032 | | M4AKHKQRKRLKSSCKRHFLYVDFSD | Functions like activin- |
| | | | VGWNDWIIAPSGYHANYCEGECPSHI | βA in cell signaling and |
| | | | AGTSGSSLSPHSTIVNHYRMRGHSPP | in vivo experiments, ~4- |
| | | | ANLKSCCVPTKLRPMSMLYYDDGQNV | fold lower potency, |
| | | | VIKITYQDMIVEECGCS | replaces TGF-beta 1 in |
| | | | FGP2. | chemically-defined stem |

What is claimed is:

1. A composition comprising
a purified chimeric polypeptide selected from the group consisting of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab; and
a purified population of adipose stromal cells.

2. The composition of claim 1, wherein the purified chimeric polypeptide comprises the sequence MQAKHKQRKRLKSSCKRHPLYVDFSDVGWNDWIIAPSGYHANYCEGECPSHIAGTSGSSL SFHSTLVNHYRMRGHSPFANLKSCCVPTKLRPMSMLYYDDGQNVVLKNYQDMIVEECGC S (SEQ ID NO: 1).

3. A method of increasing the expression of chondrogenic proteins in purified adipose stromal cells, said method comprising contacting a purified population of adipose stromal cells with a purified chimeric polypeptide selected from the group consisting of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab under conditions conducive for growth of said cells, thereby increasing expression of chondrogenic proteins.

4. The method of claim 6, wherein the chondrogenic proteins are selected from the group consisting of collagen II, aggrecan and Sox9.

5. The method of claim 6, wherein the method is carried out in vivo or in vitro.

6. The method of claim 6, wherein said method further comprises administering the compostion to a subject.

7. The method of claim 9, wherein said purified adipose stromal cells are derived from the subject.

8. The method of claim 9 wherein the composition is injected into the subject at a site where chondrocyte formation is desired.

9. The method of claim 6, wherein adipose stromal cells are contacted with AB235 having the sequence M Q A K H K Q R K R L K S S C K R H P L Y V D F S D V G W N D W I I A P S G Y H A N Y C E G E C P S H I A G T S G S S L S F H S T L V N H Y R M R G H S P F A N L K S C C V P T K L R P M S M L Y Y D D G Q N V V L K N Y Q D M I V E E C G C S (SEQ ID NO: 1).

10. The method of claim 12, wherein said contact increases type II collagen expression by about 79 fold, Sox9 expression by about 14 fold, and Aggrecan expression by about 20-fold relative to control.

11. An adipose stromal cell having increased expression of type II collagen, Sox9, and Aggrecan relative to a control, wherein the adipose stromal cell is capable of differentiating into an articular chondrogenic lineage.

12. A method for treating a subject having cartilage damage or degeneration the method comprising administering to the subject an adipose stromal cell having increased expression of type II collagen, Sox9, and Aggrecan relative to a control, wherein the adipose stromal cell is capable of differentiating into an articular chondrogenic lineage.

13. The method of claim 15, wherein the cell is injected at a site where chondrocyte formation is desired.

14. A kit for inducing the formation of chondrocytes, said kit comprising
a purified chimeric polypeptide selected from the group consisting of 1b2b3b4b5b6a, 1b2b3b4b5a6a, 1b2b3b4b5a6b, 1b2b3a4a5a6a, 1b2b3a4a5b6a, 1b2a3a4a5a6a, 1b2a3a4a5a6a L66V/V67I, 1b(1a_II)2a3a4a5a6a, 1b2a3a4a5a6b, 1b2a3a4a5b6b, 1b2a3a4a5b6a, 1b2a3b4b5b6a, 1b2a3b4b5a6a, 1b2a3b4b5a6b, and 1b2a3a4a5a6ab;
a suitable cell culture media; and
instructional material.

15. A composition comprising a mixture of
a purified chimeric polypeptide comprising the sequence of MQAKHKQRKRLKSSCKRHPLYVDFSDVGWNDWIIAPSGYHANYCEGECPSHIAGTSGSSLSF HSTLVNHYRMRGHSPFANLKSCCVPTKLRPMSMLYYDDGQNVVLKNYQDMIVEECGCS (SEQ ID NO: 1); and culture media comprising Dulbecco's modified Eagle's medium with 10% fetal bovine serum.


SEQUENCE LISTING (118 sequences)

SEQ ID NO: 1 - protein, 120 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSFHSTLVNHYRMRGHSPFANLKSCCVPTKLRPMSMLYYDDGQNVVLKNYQDMIVEECGCS

SEQ ID NO: 2 - protein, 114 aa, Homo sapiens
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - protein, 116 aa, Homo sapiens [sequence omitted]

SEQ ID NO: 4 - protein, 30 aa, Homo sapiens
QAKHKQRKRLKSSCKRHPLYVDFSDVGWND

SEQ ID NO: 5 - protein, 27 aa, Homo sapiens
GLECDGKVNICCKKQFFVSFKDIGWND

SEQ ID NO: 6 - protein, 18 aa, Homo sapiens
WIVAPPGYHAFYCHGECP

SEQ ID NO: 7 - protein, 18 aa, Homo sapiens
WIIAPSGYHANYCEGECP

SEQ ID NO: 8 - protein, 20 aa, Homo sapiens
FPLADHLNSTNHAIVQTLVN

SEQ ID NO: 9 - protein, 19 aa, Homo sapiens
SHIAGTSGSSLSFHSTVIN

SEQ ID NO: 10 - protein, 13 aa, Homo sapiens
SVNSKIPKACCVP

SEQ ID NO: 11 - protein, 19 aa, Homo sapiens
HYRMRGHSPFANLKSCCVP

SEQ ID NO: 12 - protein, 12 aa, Homo sapiens
TELSAISMLYLD

SEQ ID NO: 13 - protein, 12 aa, Homo sapiens
TKLRPMSMLYYD

SEQ ID NO: 14 - protein, 4 aa, Homo sapiens
TLVN

SEQ ID NO: 15 - protein, 4 aa, Homo sapiens
TVIN

SEQ ID NO: 16 - protein, 4 aa, Homo sapiens
LYLD

SEQ ID NO: 17 - protein, 4 aa, Homo sapiens
LYYD

SEQ ID NO: 18 - protein, 12 aa, Homo sapiens
TELSAISMLYYD

SEQ ID NO: 19 - protein, 21 aa, Homo sapiens
ENEKVVLKNYQDMVVEGCGCR

SEQ ID NO: 20 - protein, 29 aa, Homo sapiens
RGLECDGKVNICCKKQFFVSFKDIGWNDW

SEQ ID NO: 21 - protein, 19 aa, Homo sapiens
SHIAGTSGSSLSFHSTLVN

SEQ ID NO: 22 - protein, 21 aa, Homo sapiens
DGQNIIKKDIQNMIVEECGCS

SEQ ID NO: 23 - protein, 32 aa, Artificial Sequence
  Description of Artificial Sequence Synthetic polypeptide
QAKHKQRKRLKSSCKRHPLYVDFSDVGWNDII

SEQ ID NO: 24 - protein, 8 aa, Homo sapiens
IIKKDIQN

SEQ ID NO: 25 - protein, 8 aa, Artificial Sequence
  Description of Artificial Sequence Synthetic peptide
VVLKNYQD

SEQ ID NO: 26 - protein, 11 aa, Artificial Sequence
  Description of Artificial Sequence Synthetic peptide
KKQFFVSFKDI

SEQ ID NO: 27 - DNA, 24 bp, Artificial Sequence
  Description of Artificial Sequence Synthetic primer
ggactcatgaccacagtccatgcc

SEQ ID NO: 28 - DNA, 22 bp, Artificial Sequence
  Description of Artificial Sequence Synthetic primer
tcagggatgaccttgcccacag

SEQ ID NO: 29 - DNA, 20 bp, Artificial Sequence
  Description of Artificial Sequence Synthetic primer
aggatggcttccaccagtgc

SEQ ID NO: 30 - DNA, 22 bp, Artificial Sequence
  Description of Artificial Sequence Synthetic primer
tgcgtaaaagacctcaccctcc

SEQ ID NO: 31 - DNA, 19 bp, Artificial Sequence
  Description of Artificial Sequence Synthetic primer
gagacagcatgacgccgag

SEQ ID NO: 32 - DNA, 20 bp, Artificial Sequence
  Description of Artificial Sequence Synthetic primer
gcggatgctctcaatctggt

SEQ ID NO: 33 - DNA, 22 bp, Artificial Sequence
  Description of Artificial Sequence Synthetic primer
atggatgaggaaactggcaact

SEQ ID NO: 34 - DNA, 24 bp, Artificial Sequence
  Description of Artificial Sequence Synthetic primer
gccatcgacaagaacagtgtaagt

SEQ ID NO: 35 - DNA, 19 bp, Artificial Sequence
  Description of Artificial Sequence Synthetic primer
actccgagacgtggacatc

SEQ ID NO: 36 - DNA, 19 bp, Artificial Sequence
  Description of Artificial Sequence Synthetic primer
tgtaggtgacctggccgtg

SEQ ID NO: 37 - DNA, 20 bp, Artificial Sequence
  Description of Artificial Sequence Synthetic primer
agaacctcgaagacatcggc

SEQ ID NO: 38 - DNA, 21 bp, Artificial Sequence
  Description of Artificial Sequence Synthetic primer
ggctgagggttaaaggcagtg

SEQ ID NO: 39 - DNA, 360 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttctcatatagcaggcacgtccgggtcctcactgtccttccactcaacgttggtcaaccactaccgcatgcggggccatagcccctttgccaacctcaaatcgtgctgtgtcccgaccaagctgagacccatgtccatgttgtactatgatgatggtcaaaacatcatcaaaaaggacattcagaacatgatcgtggaggagtgtgggtgctca

SEQ ID NO: 40 - DNA, 360 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttctcatatagcaggcacgtccgggtcctcactgtccttacactcaacgttggtcaaccactaccgcatgcggggccatagcccctttgccaacctcaaatcgtgctgtgtcccgacagagctcagtgctatctcgatgttgtactatgatgatggtcaaaacatcatcaaaaaggacattcagaacatgatcgtggaggagtgtgggtgctca

SEQ ID NO: 41 - DNA, 360 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttctcatatagcaggcacgtccgggtcctcactgtccttccactcaacgttggtcaaccactaccgcatgcggggccatagcccctttgccaacctcaaatcgtgctgtgtcccgacagaactcagtgctatctcgatgttgtactatgatgatggtcaaaacatcatcaaaaaggacattcagaacatgatcgtggaggagtgtgggtgctca

SEQ ID NO: 42 - DNA, 345 bp, Artificial Sequence
  Description of Artificial Sequence Synthetic polynucleotide
atgcaagccaaacacaaacagcggaagcgtcttaagtccagctgcaaaaggcaccctttgtatgtggacttcagtgatgtggggtggaatgactggatcattgctccctctggctatcatgccaactactgcgacggagaatgcccttttcctctggctgatcatctgaactccactaatcatgccattgttcagacgttggtcaactctgttaactctaagattcctaaggcatgctgtgtcccgaccaagctgagacccatgtccatgttgtactatgatgatggtcaaaacatcatcaaaaaggacattcagaacatgatcgtggaggagtgtgggtgctca

SEQ ID NO: 43 - DNA, 345 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgc

SEQ ID NO: 44 - DNA, 360 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttctcatatagcaggcacgtccgggtcctcactgtccttccactcaacgttggtcaaccactaccgcatgcggggccatagcccctttgccaacctcaaatcgtgctgtgtcccgacagaactcagtgctatctcgatgttgtaccttgacgagaatgaaaaggttgtattaaagaactatcaggacatggttgtggagggttgtgggtgtcgc

SEQ ID NO: 45 - DNA, 360 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttctcatatagcaggcacgtccgggtcctcactgtccttccactcaacgttggtcaaccactaccgcatgcggggccatagcccctttgccaacctcaaatcgtgctgtgtcccgaccaagctgagacccatgtccatgttgtaccttgacgagaatgaaaaggttgtattaaagaactatcaggacatggttgtggagggttgtgggtgtcgc

SEQ ID NO: 46 - DNA, 360 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttcccatatagcaggcacgtccgggtcctcactgtccttccattcaacgttggtcaaccactaccgcatgcggggccatagcccctttgccaacctcaaatcgtgctgtgtcccgaccaagctgagacccatgtccatgttgtactatgatgatggtcaaaacatcatcaaaaaggacattcagaacatgatcgtggaggagtgtgggtgctca

SEQ ID NO: 47 - DNA, 360 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttcccatatagcaggcacgtccgggtcctcactgtccttccattcaacggtgatcaaccactaccgcatgcggggccatagcccctttgccaacctcaaatcgtgctgtgtcccgaccaagctgagacccatgtccatgttgtactatgatgatggtcaaaacatcatcaaaaaggacattcagaacatgatcgtggaggagtgtgggtgctca

SEQ ID NO: 48 - DNA, 357 bp, Artificial Sequence
  Description of Artificial Sequence Synthetic polynucleotide
atgcaagccaaacacaaacagcggaaacgccttaagtccagctgtaagaaacagttctttgtcagtttcaaggacatcgggtggaatgactggatcattgctccctctggctatcatgccaactactgcgagggagaatgcccttcccatatagcaggcacgtccgggtcctcactgtccttccattcaacgttggtcaaccactaccgcatgcggggccatagcccctttgccaacctcaaatcgtgctgtgtcccgaccaagctgagacccatgtccatgttgtactatgatgatggtcaaaacatcatcaaaaaggacattcagaacatgatcgtggaggagtgtgggtgctca

SEQ ID NO: 49 - DNA, 342 bp, Artificial Sequence
  Description of Artificial Sequence Synthetic polynucleotide
atgcaagccaaacaccaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgaccaagctgagaccctccatgttgtactatgatgatggtcaaaacatcatcaaaaaggacattcagaacatgatcgtggaggagtgtgggtgctca

SEQ ID NO: 50 - DNA, 345 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgttgtactatgatgatggtcgaaacatcatcaaaaaggacattcagaacatgatcgtggaggagtgtgggtgctca

SEQ ID NO: 51 - DNA, 345 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgaccaagctgagacccatgtccatgttgtactatgatgagaatgaaaaggttgtattaaagaactatcaggacatggttgtggagggttgtgggtgtcgc

SEQ ID NO: 52 - DNA, 345 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgacggagaatgcccttttcctctggctgatcatctgaactccactaatcatgccattgttcagacgttggtcaactctgttaactctaagattcctaaggcatgctgtgtcccgaccaagctgagacccatgtccatgttgtaccttgacgagaatgaaaaggttgtattaaagaactatcaggacatggttgtggagggttgtgggtgtcgc

SEQ ID NO: 53 - DNA, 345 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgacggagaatgcccttttcctctggctgatcatctgaactccactaatcatgccattgttcagacgttggtcaactctgttaactctaagattcctaaggcatgctgtgtcccgacagaactcagtgctatctcgatgttgtactatgatgatggtcaaaacatcatcaaaaaggacattcagaacatgatcgtggaggagtgtgggtgctca

SEQ ID NO: 54 - DNA, 345 bp, Artificial Sequence
  Description of Artificial Sequence Synthetic polynucleotide
atgcaagccaaacacaaacagcggaagcgtcttaagtccagctgcaaaaggcaccctttgtatgtggacttcagtgatgtggggtggaatgactggatcattgctccctctggctatcatgccaactactgcgagggagaatgcccttttcctctggctgatcatctgaactccactaatcacgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgcgggtgtcgt

SEQ ID NO: 55 - DNA, 363 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaaccactaccgcatgcggggccatagcccctttgccaacctcaaatcgtgctgtgtcccgaccaagctgagacccatgtccatgttgtactatgatgatggtcaaaacatcatcaaaaaggacattcagaacatgatcgtggaggagtgtgggtgctca

SEQ ID NO: 56 - DNA, 363 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaaccactaccgcatgcggggccatagcccctttgccaacctcaaatcgtgctgtgtcccgacagaactcagtgctatctcgatactgtaccttgacgagaatgaaaaggttgtattaaagaactatcaggacatggttgtggagggttgtgggtgtcgc

SEQ ID NO: 57 - DNA, 363 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaaccactaccgcatgcggggccatagcccctttgccaacctcaaatcgtgctgtgtcccgaccaagctgagacccatgtccatgttgtactatgatgagaatgaaaaggttgtattaaagaactatcaggacatggttgtggagggttgcgggtgtcgt

SEQ ID NO: 58 - DNA, 363 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaaccactaccgcatgcggggccatagcccctttgccaacctcaaatcgtgctgtgtcccgacagaactcagtgctatctcgatgttgtactatgatgatggtcaaaacatcatcaaaaaggacattcagaacatgatcgtggaggagtgtgggtgctca

SEQ ID NO: 59 - DNA, 360 bp, Artificial Sequence
  Description of Artificial Sequence Synthetic polynucleotide
atgcaagccaaacacaaacagcggaaacgccttaagtccagctgtaagagacaccctttgtatgtggacttcagtgacgtggggtggaatgactggattgtggctcccccggggtatcacgccttttactgccacggagaatgcccttctcatatagcaggcacgtccgggtcctcactgtccttccactcaacgttggtcaaccactaccgcatgcggggccatagcccctttgccaacctcaaatcgtgctgtgtcccgaccaagctgagacccatgtccatgttgtaccttgacgagaatgaaaaggttgtattaaagaactatcaggacatggttgtggagggttgtgggtgtcgc

SEQ ID NO: 60 - DNA, 351 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttctcatatagcaggcacgtccgggtcctcactgtccttccactcaacgttggtcaaccactaccgcatgcggggccatagcccctttgccaacctcaaatcgtgctgtgtcccgacagaactcagtgctatctcgatgttgtactatgatgagaatgaaaaggttgtattaaagaactatcaggacatggttgtggagggttgc

SEQ ID NO: 61 - DNA, 342 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttctcatatagcaggcacgtccgggtcctcactgtccttccactcaacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgcgggtgtcgt

SEQ ID NO: 62 - DNA, 342 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttctcatatagcaggcacgtccgggtcctcactgtccttccactcaacgttggtcaactctgttaactctaagattcctaaggcatgctgtgtcccgacagaactcagtgctatctcgatgttgtactatgatgatggtcaaaacatcatcaaaaaggacattcagaacatgatcgtggaggagtgtgggtgctca

SEQ ID NO: 63 - DNA, 342 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttctcatatagcaggcacgtccgggtcctcactgtccttccactcaacgttggtcaactctgttaactctaagattcctaaggcatgctgtgtcccgaccaagctgagacccatgtccatgttgtactatgatgatggtcaaaacatcatcaaaaaggacattcagaacatgatcgtggaggagtgtgggtgctca

SEQ ID NO: 64 - DNA, 342 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttctcatatagcaggcacgtccgggtcctcactgtccttccactcaacgttggtcaactctgttaactctaagattcctaaggcatgctgtgtcccgaccaagctgagacccatgtccatgttgtactatgatgagaatgaaaaggttgtattaaagaactatcaggacatggttgtggagggttgcgggtgtcgt

SEQ ID NO: 65 - DNA, 342 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttctcatatagcaggcacgtccgggtcctcactgtccttccactcaacgttggtcaactctgttaactctaagattcctaaggcatgctgtgtcccgacagaactcagtgctatctcgatgttgtaccttgacgagaatgaaaaggttgtattaaagaactatcaggacatggttgtggagggttgtgggtgtcgc

SEQ ID NO: 66 - DNA, 342 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttctcatatagcaggcacgtccgggtcctcactgtccttccactcaacgttggtcaactctgttaactctaagattcctaaggcatgctgtgtcccgacagaactcaatgctatctcgatgttgtactatgatgatggtcaaaacatcattaaaaaggacattcagaacatgatcgtggaggagtgtgggtgctca

SEQ ID NO: 67 - DNA, 342 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttctcatatagcaggcacgtccgggtcctcactgtccttccactcaacgttggtcaactctgttaactctaagattcctaaggcatgctgtgtcccgaccaagctgagacccatgtccatgttgtactatgatgatggtcaaaacatcatcaaaaaggacattcagaacatgatcgtggaggagtgtgggtgctca

SEQ ID NO: 68 - DNA, 363 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttttcctctggctgatcatctgaactccactaatcatgccattgttcagacgttggtcaaccactaccgcatgcggggccatagcccctttgccaacctcaaatcgtgctgtgtcccgaccaagctgagacccatgtccatgttgtactatgatgatggtcaaaacatcatcaaaaaggacattcagaacatgatcgtggaggagtgtgggtgctca

SEQ ID NO: 69 - DNA, 363 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttttcctctggctgatcatctgaactctactaatcatgccattgttcagacgttggtcaaccactaccgcatgcggggccatagcccctttgccaacctcaaatcgtgctgtgtcccgacagaactcagtgctatctcgatgctgtaccttgacgatggtcaaaacatcatcaaaaagcacattcagaacatgatcgtggaggagtgtgggtgctca

SEQ ID NO: 70 - DNA, 363 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggatcattgctccctctggctatcatgccaactactgcgagggagaatgcccttttcctctggctgatcatctgaactctactaatcatgccattgttcagacgttggtcaaccactaccgcatgcggggccatagcccctttgccaacctcaaatcgtgctgtgtcccgacagaactcagtgctatctcgatgctgtaccttgacgagaatgaaaaggttgtattaaagaactatcaggacatggttgtggagggttgcgggtgtcgt

SEQ ID NO: 71 - DNA, 363 bp, Artificial Sequence
  Description of Artificial Sequence Synthetic polynucleotide
atgcaagccaaacacaaacagcggaagcgtcttaagtccagctgcaaaaggcaccctttgtatgtggacttcagtgatgtggggtggaatgactggatcattgctccctctggctatcatgccaactactgcgacggagaatgcccttttcctctggctgatcatctgaactccactaatcatgccattgttcagacgttggtcaaccactaccgcatgcggggccatagcccctttgccaacctcaaatcatgctgtgtcccgaccaagctgagacccatgtccatgttgtactatgatgagaatgaaaaggttgtattaaagaactatcaggacatggttgtggagggttgcgggtgtcgt

SEQ ID NO: 72 - DNA, 348 bp, Artificial Sequence
  Description of Artificial Sequence Synthetic polynucleotide
atggctcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgc

SEQ ID NO: 73 - DNA, 344 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattatcgcgcctgaaggctacgccgcctactactgtgagggggagtgtgccttccctctgaactcctacatgaacgccaccaaccacgccatcgtgcagacgctggtccacttcatcaacccggaacggtgcccaagtgctgtgcgcccacgcagctcaatgccatctccgtcctctacttcgatgacagctccaacgtcatcctgaagaaatacagaaacatggtggtccgggcctgtggctgccac

SEQ ID NO: 74 - DNA, 351 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattattgccccaaaagagtacgaggcatacgagtgtaagggcggctgtttctttccgctggccgacgatgtcaccccgaccaagcacgcaattgtccaaaccttagtgcacctgaagttcccaacgaaagtgggtaaggcatgttgtgtgccaaccaagttatctccaattagcgtgctgtataaggatgatatgggcgtgccgacgttaaagtatcattacgagggcatgagcgtcgcagagtgtggctgccgc

SEQ ID NO: 75 - DNA, 348 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattatcgcgccgctggactacgaggcgtaccactgcgagggcctatgcgattttcctctgcgttcgcacctcgaacccaccaaccatgccatcattcagacgttggtcaactccatggcaccagacgcggcgccggcctcctgctgtgtcccggcgcgcctcagccccatcagcatcttgtactatgatgccgccaacaacgttgtctacaagcaatacgaggacatggtggtggaggcctgtgggtgtcgc

SEQ ID NO: 76 - DNA, 330 bp, Artificial Sequence
  Description of Artificial Sequence Synthetic polynucleotide
atgcaagccaaacacaaacagcggaaacgccttaagtccagctgtaagagacaccctttgtacgtggacttcagtgacgtggggtggaatgactggattattgcacccaaaagatataaggccaattactgctctggagagtgtgaatttgtatttttacaaaaataccctcacactcatcttgtgcaccaagcaaaccccagaggttcagcaggcccctgctgtactcccacaaagatgtctccaatcaatatgctatattttaatggcaaagaacaaataatatatgggaaaattccagccatggtagtagatcgctgtgggtgctca

SEQ ID NO: 77 - DNA, 399 bp, Artificial Sequence
  Description of Artificial Sequence Synthetic polynucleotide
atgcaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccac

SEQ ID NO: 78 - protein, 120 aa, Artificial Sequence
  Description of Artificial Sequence Synthetic polypeptide
MQAKHKQRKRLKSSCKRHPLYVDFSDVGWNDWIVAPPGYHAFYCHGECPSHIAGTSGSSLSFHSTLVNHYRMRGHSPFANLKSCCVPTKLRPMSMLYYDDGQNIIKKDIQNMIVEECGCS

SEQ ID NO: 79 - protein, 120 aa, Artificial Sequence
  Description of Artificial Sequence Synthetic polypeptide
MQAKHKQRKRLKSSCKRHPLYVDFSDVGWNDWIVAPPGYHAFYCHGECPSHIAGTSGSSLSLHSTLVNHYRMRGHSPFANLKSCCVPTELSAISMLYYDDGQNIIKKDIQNMIVEECGCS

SEQ ID NO: 80 - protein, 120 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSFHSTLVNHYRMRGHSPFANLKSCCVPTELSAISMLYYDDGQNIIKKDIQNMIVEECGCS

SEQ ID NO: 81 - protein, 115 aa, Artificial Sequence
  Description of Artificial Sequence Synthetic polypeptide
MQAKHKQRKRLKSSCKRHPLYVDFSDVGWNDWIIAPSGYHANYCDGECPFPLADHLNSTNHAIVQTLVNSVNSKIPKACCVPTKLRPMSMLYYDDGQNIIKKDIQNMIVEECGCS

SEQ ID NO: 82 - protein, 115 aa, Artificial Sequence
  Description of Artificial Sequence Synthetic polypeptide
MQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 83 - protein, 120 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSFHSTLVNHYRMRGHSPFANLKSCCVPTELSAISMLYLDENEKVVLKNYQDMVVEGCGCR

SEQ ID NO: 84 - protein, 120 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSFHSTLVNHYRMRGHSPFANLKSCCVPTKLRPMSMLYLDENEKVVLKNYQDMVVEGCGCR

SEQ ID NO: 85 - protein, 120 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSFHSTLVNHYRMRGHSPFANLKSCCVPTKLRPMSMLYYDDGQNIIKKDIQNMIVEECGCS

SEQ ID NO: 86 - protein, 120 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 87 - protein, 119 aa, Artificial Sequence
  Description of Artificial Sequence Synthetic polypeptide
MQAKHKQRKRLKSSCKKQFFVSFKDIGWNDWIIAPSGYHANYCEGECPSHIAGTSGSSLSFHSTLVNHYRMRGHSPFANLKSCCVPTKLRPMSMLYYDDGQNIIKKDIQNMIVEECGCS

SEQ ID NO: 88 - protein, 114 aa, Artificial Sequence
  Description of Artificial Sequence Synthetic polypeptide
MQAKHQQRKRLKSSCKRHPLYVDFSDVGWNDWIVAPPGYHAFYCHGECPFPLADHLNSTNHAIVQTLVNSVNSKIPKACCVPTKLRPSMLYYDDGQNIIKKDIQNMIVEECGCS

SEQ ID NO: 89 - protein, 115 aa, Artificial Sequence
  Description of Artificial Sequence Synthetic polypeptide
MQAKHKQRKRLKSSCKRHPLYVDFSDVGWNDWIVAPPGYHAFYCHGECPFPLADHLNSTNHAIVQTLVNSVNSKIPKACCVPTELSAISMLYYDDGRNIIKKDIQNMIVEECGCS

SEQ ID NO: 90 - protein, 115 aa, Artificial Sequence
  Description of Artificial Sequence Synthetic polypeptide
MQAKHKQRKRLKSSCKRHPLYVDFSDVGWNDWIVAPPGYHAFYCHGECPEPLADHLNSTNHAIVQTLVNSVNSKIPKACCVPTKLRPMSMLYYDENEKVVLKNYQDMVVEGCGCR

SEQ ID NO: 91 - protein, 115 aa, Artificial Sequence
  Description of Artificial Sequence Synthetic polypeptide
MQAKHKQRKRLKSSCKRHPLYVDFSDVGWNDWIIAPSGYHANYCDGECPFPLADHLNSTNHAIVQTLVNSVNSKIPKACCVPTKLRPMSMLYLDENEKVVLKNYQDMVVEGCGCR

SEQ ID NO: 92 - protein, 115 aa, Artificial Sequence
  Description of Artificial Sequence Synthetic polypeptide
MQAKHKQRKRLKSSCKRHPLYVDFSDVGWNDWIIAPSGYHANYCDGECPFPLADHLNSTNHAIVQTLVNSVNSKIPKACCVPTELSAISMLYYDDGQNIIKKDIQNMIVEECGCS

SEQ ID NO: 93 - protein, 115 aa, Artificial Sequence
  Description of Artificial Sequence Synthetic polypeptide
MQAKHKQRKRLKSSCKRHPLYVDFSDVGWNDWIIAPSGYHANYCEGECPFPLADHLNSTNHAIVQTLVNSVNSKIPKACCVPTELSAISMLYLDENEKVVLKNYQDMVVEGCGCR

SEQ ID NO: 94 - protein, 121 aa, Artificial Sequence
  Description of Artificial Sequence Synthetic polypeptide
MQAKHKQRKRLKSSCKRHPLYVDFSDVGWNDWIVAPPGYHAFYCHGECPFPLADHLNSTNHAIVQTLVNHYRMRGHSPFANLKSCCVPTKLRPMSMLYYDDGQNIIKKDIQNMIVEECGCS

SEQ ID NO: 95 - protein, 121 aa, Artificial Sequence
  Description of Artificial Sequence Synthetic polypeptide
MQAKHKQRKRLKSSCKRHPLYVDFSDVGWNDWIVAPPGYHAFYCHGECPFPLADHLNSTNHAIVQTLVNHYRMRGHSPFANLKSCCVPTELSAISILYLDENEKVVLKNYQDMVVEGCGCR

SEQ ID NO: 96 - protein, 121 aa, Artificial Sequence
  Description of Artificial Sequence Synthetic polypeptide
MQAKHKQRKRLKSSCKRHPLYVDFSDVGWNDWIVAPPGYHAFYCHGECPFPLADHLNSTNHAIVQTLVNHYRMRGHSPFANLKSCCVPTKLRPMSMLYYDENEKVVLKNYQDMVVEGCGCR

SEQ ID NO: 97 - protein, 121 aa, Artificial Sequence
  Description of Artificial Sequence Synthetic polypeptide
MQAKHKQRKRLKSSCKRHPLYVDFSDVGWNDWIVAPPGYHAFYCHGECPFPLADHLNSTNHAIVQTLVNHYRMRGHSPFANLKSCCVPTELSAISMLYYDDGQNIIKKDIQNMIVEECGCS

SEQ ID NO: 98 - protein, 120 aa, Artificial Sequence
  Description of Artificial Sequence Synthetic polypeptide
MQAKHKQRKRLKSSCKRHPLYVDFSDVGWNDWIVAPPGYHAFYCHGECPSHIAGTSGSSLSFHSTLVNHYRMRGHSPFANLKSCCVPTKLRPMSMLYLDENEKVVLKNYQDMVVEGCGCR

SEQ ID NO: 99 - protein, 120 aa, Artificial Sequence
  Description of Artificial Sequence Synthetic polypeptide
MQAKHKQRKRLKSSCKRHPLYVDFSDVGWNDWIVAPPGYHAFYCHGECPSHIAGTSGSSLSFHSTLVNHYRMRGHSPFANLKSCCVPTELSAISMLYLDENEKVVLKNYQDMVVEGCGCR

SEQ ID NO: 100 - protein, 114 aa, Artificial Sequence
  Description of Artificial Sequence Synthetic polypeptide
MQAKHKQRKRLKSSCKRHPLYVDFSDVGWNDWIVAPPGYHAFYCHGECPSHIAGTSGSSLSFHSTLVNSVNSKIPKACCVPTELSAISMLYLDENEKVVLKNYQDMVVEGCGCR

SEQ ID NO: 101 - protein, 114 aa, Artificial Sequence
  Description of Artificial Sequence Synthetic polypeptide
MQAKHKQRKRLKSSCKRHPLYVDFSDVGWNDWIVAPPGYHAFYCHGECPSHIAGTSGSSLSFHSTLVNSVNSKIPKACCVPTELSAISMLYYDDGQNIIKKDIQNMIVEECGCS

SEQ ID NO: 102 - protein, 114 aa, Artificial Sequence
  Description of Artificial Sequence Synthetic polypeptide
MQAKHKQRKRLKSSCKRHPLYVDFSDVGWNDWIVAPPGYHAFYCHGECPSHIAGTSGSSLSFHSTLVNSVNSKIPKACCVPTKLRPMSMLYYDDGQNIIKKDIQNMIVEECGCS

SEQ ID NO: 103 - protein, 114 aa, Artificial Sequence
  Description of Artificial Sequence Synthetic polypeptide
MQAKHKQRKRLKSSCKRHPLYVDFSDVGWNDWIVAPPGYHAFYCHGECPSHIAGTSGSSLSFHSTLVNSVNSKIPKACCVPTKLRPMSMLYYDENEKVVLKNYQDMVVEGCGCR

SEQ ID NO: 104 - protein, 114 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSPHSTLVNSVNSKIPKACCVPTELSAISMLYLDENEKVVLKNYQDMVVEGCGCR

SEQ ID NO: 105 - protein, 114 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSFHSTLVNSVNSKIPKACCVPTELNAISMLYYDDGQNIIKKDIQNMIVEECGCS

SEQ ID NO: 106 - protein, 114 aa, Artificial Sequence
  Description of Artificial Sequence Synthetic polypeptide
MQAKHKQRKRLKSSCKRHPLYVDFSDVGWNDWIIAPSGYHANYCEGECPSHIAGTSGSSLSFHSTLVNSVNSKIPKACCVPTKLRPMSMLYYDDGQNIIKKDIQNMIVEECGCS

SEQ ID NO: 107 - protein, 121 aa, Artificial Sequence
  Description of Artificial Sequence Synthetic polypeptide
MQAKHKQRKRLKSSCKRHPLYVDFSDVGWNDWIIAPSGYHANYCEGECPFPLADHLNSTNHAIVQTLVNHYRMRGHSPFANLKSCCVPTKLRPMSMLYYDDGQNIIKKDIQNMIVEECGCS

SEQ ID NO: 108 - protein, 121 aa, Artificial Sequence
  Description of Artificial Sequence Synthetic polypeptide
MQAKHKQRKRLKSSCKRHPLYVDFSDVGWNDWIIAPSGYHANYCEGECPFPLADHLNSTNHAIVQTLVNHYRMRGHSPFANLKSCCVPTELSAISMLYYDDGQNIIKKDIQNMIVEECGCS

SEQ ID NO: 109 - protein, 121 aa, Artificial Sequence
  Description of Artificial Sequence Synthetic polypeptide
MQAKHKQPKRLKSSCKRHPLYVDFSDVGWNDWIIAPSGYHANYCEGECPFPLADHLNSTNHAIVQTLVNHYRMRGHSPFANLKSCCVPTELSAISMLYLDENEKVVLKNYQDMVVEGCGCR

SEQ ID NO: 110 - protein, 121 aa, Artificial Sequence
  Description of Artificial Sequence Synthetic polypeptide
MQAKHKQRKRLKSSCKRHPLYVDFSDVGWNDWIIAPSGYHANYCDGECPFPLADHLNSTNHAIVQTLVNHYRMRGHSPFANLKSCCVPTKLRPMSMLYYDENEKVVLKNYQDMVVEGCGCR

SEQ ID NO: 111 - protein, 116 aa, Artificial Sequence
  Description of Artificial Sequence Synthetic polypeptide
MAQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 112 - protein, 116 aa, Artificial Sequence
  Description of Artificial Sequence Synthetic polypeptide
MQAKHKQRKRLKSSCKRHPLYVDFSDVGWNDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 113 - protein, 117 aa, Artificial Sequence
  Description of Artificial Sequence Synthetic polypeptide
MQAKHKQRKRLKSSCKRHPLYVDFSDVGWNDWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 114 - protein, 116 aa, Artificial Sequence
  Description of Artificial Sequence Synthetic polypeptide
MQAKHKQRKRLKSSCKRHPLYVDFSDVGWNDWIIAPLDYEAYHCEGLCDFPLRSHLEPTNHAIIQTLVNSMAPDAAPASCCVPARLSPISILYYDAANNVVYKQYEDMVVEACGCR

SEQ ID NO: 115 - protein, 109 aa, Artificial Sequence
  Description of Artificial Sequence Synthetic polypeptide
MQAKHKQRKRLKSSCKRHPLYVDFSDVGWNDWIIAPKRYKANYCSGECEFVFLQKYPHTHLVHQANPRGSAGPCCTPTKMSPINMLYFNGKEQIIYGKPAMVVDRCGCS

SEQ ID NO: 116 - protein, 133 aa, Artificial Sequence
  Description of Artificial Sequence Synthetic polypeptide
MQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 117 - protein, 120 aa, Artificial Sequence; identical to SEQ ID NO: 1
  Description of Artificial Sequence Synthetic polypeptide

SEQ ID NO: 118 - protein, 4 aa, Homo sapiens
QAKH